(19) United States
(12) Patent Application Publication
(10) Pub. No.: US 2021/0220074 A1
(43) Pub. Date: Jul. 22, 2021

(54) SURGICAL TOOL SUPPORT SYSTEMS INCLUDING FRAME MOUNT ASSEMBLIES AND RELATED METHODS

(71) Applicant: ClearPoint Neuro, Inc., Irvine, CA (US)

(72) Inventors: David John Sayler, Portland, OR (US); Peter G. Piferi, Orange, CA (US); Rajesh Pandey, Irvine, CA (US); Maxwell Jerad Daly, Redlands, CA (US)

(21) Appl. No.: 17/149,884

(22) Filed: Jan. 15, 2021

Related U.S. Application Data
(60) Provisional application No. 62/964,340, filed on Jan. 22, 2020.

(51) Int. Cl. A61B 90/14; A61B 34/20; A61B 90/11; G01R 33/30

(52) U.S. Cl. CPC A61B 90/14; A61B 34/20; A61B 2090/374; G01R 33/30; A61B 2034/2051; A61B 90/11

(57) ABSTRACT

Systems and assemblies for use during image-guided procedures have surgical tool support systems with adjustable attachment locations provided by fixed support structures and cooperating coupling arms attached thereto that provide different lockable orientations accommodating both supine and occipital access for neurological procedures.

### RELATED APPLICATIONS

This patent application claims the benefit of and priority to U.S. Provisional Application Ser. No. 62/964,340, filed Jan. 22, 2020, the contents of which are hereby incorporated by reference as if recited in full herein.

### FIELD OF THE INVENTION

The present invention relates generally to devices used during medical procedures and may be particularly suitable for use in MM-guided procedures.

### BACKGROUND OF THE INVENTION

Image guided procedures such as MM guided interventional procedures are becoming more viable and may provide improved outcomes, alternative procedures and/or therapies over conventional procedures.

### SUMMARY OF EMBODIMENTS OF THE INVENTION

Embodiments of the invention are directed to surgical devices that can provide external structural support for intrabody surgical devices. The devices may be configured for CT or MRI environments or may be configured to be compatible for both CT and MRI environments.

Embodiments of the invention provide a surgical tool support system with a surgical frame mount assembly that provides selectable lockable orientations, including a first orientation for a supine procedure and a second orientation for an occipital procedure.

A base of the frame mount assembly in the second orientation can be at an angle that is in a range of 15-60 degrees from horizontal.

A surgical tool, such as a trajectory guide, can be moveably attached to an arm of the surgical frame mount assembly so that the surgical tool can be positionally adjusted relative to a target anatomical location of a patient.

The systems can include a frame mount assembly with coupling arms that couple to a table mount assembly. The frame mount assembly can include a frame mount member, a base and an arm coupled to the base and the frame mount member. The frame mount member, base and arm are pivotable as a unit relative to table mount assembly and/or a head fixation member between the first and second lockable orientations.

The surgical tool can be a trajectory guide or can be held by a trajectory guide. The trajectory guide can include a trajectory guide base with an elongate slot. A mounting member can extend through the elongate slot of the trajectory guide to attach the trajectory guide to the arcuate segment of the at least one turret arm so that the trajectory guide can be positionally adjustable relative to a patient, in X, Y and Z directions.

Embodiments of the invention are directed to surgical tool support systems that include: a frame member comprising a perimeter with a top, bottom and right and left sides surrounding an open space; a base coupled to the bottom of the frame member; at least one support arm having opposing first and second ends, the first end coupled to the top of the frame member, the second end coupled to the base, wherein the at least one support arm extends across the open space between the top and the bottom of the frame member; and first and second coupling arms, the first coupling arm attached to the right side of the frame member, the second coupling arm attached to the left side of the frame member. The frame member, the base and the at least one support arm are configured to move as a unit between one of a plurality of different use orientations to thereby allow supine and occipital procedures.

The system can include a first side support member attached to a first table mount assembly and a second side support member attached to a second table mount assembly. The first coupling arm can be attached (directly or indirectly) to the first side support member and the second coupling arm can be attached (directly or indirectly) to the second side support member.

The first and second side support members can be first and second orientation plates that can each have a primary channel extending upwardly and a plurality of secondary channels extending from the primary channel and being spaced apart in a vertical direction.

The base can be arcuate and can arc outward in a direction that is longitudinally spaced apart from a bottom of the frame member. The base can have an arcuate laterally extending groove that extends across an upper portion thereof. A lower end portion of the support arm can be adjustably lockable to the base.

The at least one support arm can be pivotably coupled to a medial segment of the top of the frame member.

The system can also include a trajectory guide assembly attached to the at least one support arm. The trajectory guide assembly can have a trajectory frame with a pair of adjacent legs on opposing sides of a slot that is slidably positionable at different positions above and/or below the at least one support arm.

The at least one support arm can be provided as first and second support arms each separately positionable relative to each other and the base and each can be coupled to a common attachment location at the top of the frame member.

The first support arm can have an upper portion with a downwardly extending recess. The second support arm can have an upper portion with an upwardly extending recess. The first and second support arms can cooperate to allow the first support arm to slide (rotate about) over the second support arm.

The secondary channels can have a closed end and an opposing open end. The open end can be adjacent to and merges into the primary channel.

The primary channel can be bounded by a wall that is arcuate in a vertical/height dimension.

The wall has upper and lower ends that extend distally further than a medial segment thereof.

The plurality of secondary channels are provided as between 3 and 30 secondary channels.

Neighboring secondary channels of the plurality of secondary channels are spaced apart in a vertical direction a distance in a range of about 0.1 inch to 1 inch.

The first and second coupling arms, where used, can each have a first end portion and a laterally spaced apart second end portion. The first end portion of the first coupling member can be attached to the first side of the frame member and the first end portion of the second coupling member can be attached to the second side of the frame member. The second end portion of the first coupling member can be attached to at least one laterally extending attachment member. Each of the at least one laterally extending attachment member can also extend through one of the secondary channels of the first orientation plate. The second end portion of the second coupling member can be attached to at least one laterally extending attachment member, each of which can also extend through one of the secondary channels of the second orientation plate.

Other embodiments are directed to methods for positioning a surgical tool about a head of a patient. The methods include providing a frame mount assembly comprising a frame member having a perimeter with a top, bottom and right and left sides surrounding an open space and at least one support arm that extends across the open space between the top and the bottom of the frame member. The open space is larger laterally and vertically than the head of the patient. The methods include moving the at least one support arm and the frame member as a unit between one of a plurality of different use orientations. The frame mount assembly is configured to allow a user to select at least one orientation for a supine procedure and/or at least one orientation for an occipital procedure.

The frame mount assembly can further include a base coupled to the bottom of the frame member. The at least one support member can have a first end portion attached to a medial segment of the top of the frame member and a second end portion attached to the base. The method can further include pivoting the first end portion relative to the medial segment of the top of the frame while sliding the second end portion over the base and locking the second end portion in a desired orientation relative to the base before or after the moving step.

The frame mount assembly can have left and right coupling arms that attach to left side and right side support members that can be coupled to a table mount assembly. The moving step can include mounting the left and right coupling arms to a different coupling aperture or channel of the left side and right side support members.

The method can include adjusting a position of a trajectory guide frame attached to the at least one support arm before, during or after the moving step.

The method can include attaching a head fixation frame assembly to a table mount assembly and attaching the frame member to the table mount assembly adjacent the head fixation frame assembly before the moving step.

The method can include placing multiple intrabody cannulas during a single treatment session for neurological treatment of a brain of the patient during an Mill and/or CT-image guided medical procedure.

Further features, advantages and details of the present invention will be appreciated by those of ordinary skill in the art from a reading of the figures and the detailed description of the preferred embodiments that follow, such description being merely illustrative of the present invention. Features described with respect with one embodiment can be incorporated with or into other embodiments although not specifically discussed therewith. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail in the specification set forth below.

### BRIEF DESCRIPTION OF THE DRAWINGS

FIG. 1A is a top perspective view of a surgical tool support system having a surgical frame mount assembly coupled to a head fixation assembly in a first orientation relative to a patient according to embodiments of the present invention.

FIG. 1B is a side view of the system shown in FIG. 1A according to embodiments of the present invention.

FIG. 1C is an end perspective view of the system shown in FIG. 1A and shown attached to a patient support surface according to embodiments of the present invention.

FIG. 2A is a top perspective view of the surgical tool support system shown in FIG. 1A but with the surgical frame mount assembly but positioned in a second orientation relative to a patient according to embodiments of the present invention.

FIG. 2B is a side view of the system shown in FIG. 1A with the surgical frame mount assembly in the position shown in FIG. 2A.

FIG. 3 is a top, proximal end perspective view of the surgical frame mount assembly shown in FIG. 1A according to embodiments of the present invention.

FIG. 4 is a top, distal end perspective view of the surgical frame mount assembly shown in FIG. 3 according to embodiments of the present invention.

FIG. 5A is a top perspective view of the head fixation assembly shown in FIG. 1A illustrating a patient head in position during a supine procedure according to embodiments of the present invention.

FIG. 5B is a side view of the head fixation assembly shown in FIG. 5A.

FIG. 6A is a top perspective view of the head fixation assembly shown in FIG. 1A illustrating a patient head in position during an occipital procedure according to embodiments of the present invention.

FIG. 6B is a side view of the head fixation assembly shown in FIG. 6A.

FIG. 7A is a partial, side perspective view of the head fixation assembly illustrating frame mount attachment points according to embodiments of the present invention.

FIG. 7B is a partial, side perspective view of the head fixation assembly coupled to the surgical frame mount assembly in a first orientation for a supine procedure according to embodiments of the present invention.

FIG. 7C is a partial, side perspective view of the head fixation assembly coupled to the surgical frame mount assembly in a second orientation for an occipital procedure according to embodiments of the present invention.

FIG. 8A is a side view of another embodiment of a side member of a head fixation assembly according to embodiments of the present invention.

FIGS. 8B-8C are side views of example embodiments of coupling arms of the frame mount assembly according to embodiments of the present invention.

FIGS. 9A and 9B are partial, enlarged, perspective side views of the coupling arm shown in FIG. 8D in different orientations using selected attachment points of the head fixation assembly according to embodiments of the present invention.

FIG. 9C is a partial, enlarged side perspective view of the coupling arm shown in FIG. 8C coupled to the head fixation assembly according to embodiments of the present invention

FIGS. 10A and 10B are side views of the surgical support system in the first orientation (associated with a supine position of the patient) and illustrating medial maximum (FIG. 10A) and minimum (FIG. 10B) medial positions of a surgical tool held by the surgical frame mount assembly in respective max and min positions.

FIGS. 11A and 11B are side views of the surgical support system in the first orientation shown in FIGS. 10A and 10B illustrating medial maximum (FIG. 11A) and minimum (FIG. 11B) lateral positions of a surgical tool held by the surgical frame mount assembly in respective max and min positions.

FIGS. 12A and 12B are side views of the surgical support system in the second orientation (associated with an occipital position of the patient) and illustrating medial maximum (FIG. 12A) and minimum (FIG. 12B) medial positions of a surgical tool held by the surgical frame mount assembly in respective max and min positions.

FIGS. 13A and 13B are side views of the surgical support system in the second orientation shown in FIGS. 12A and 12B illustrating lateral maximum (FIG. 13A) and minimum (FIG. 13B) lateral positions of a surgical tool held by the surgical frame mount assembly in respective max and min positions.

FIG. 14 is a flow chart of example methods of positioning a surgical tool about a head of a patient for a surgical procedure according to embodiments of the present invention.

FIGS. 15A-15P are schematic illustrations of the surgical tool system in an example workflow of an example neurological procedure according to embodiments of the present invention.

FIG. 16A is a top perspective view of another embodiment of a surgical tool support system having a surgical frame mount assembly coupled to a head fixation assembly in a first orientation relative to a patient according to embodiments of the present invention.

FIG. 16B illustrates the system of FIG. 16A in a different orientation according to embodiments of the present invention.

FIG. 17 is an enlarged side perspective view of the table mount shown in FIG. 16A.

FIG. 18 is an enlarged side perspective view of the frame mount shown in FIG. 16A.

FIG. 19 is an enlarged side view of the angling plate shown in FIG. 16A.

FIG. 20 is a side perspective view of a base for a trajectory guide assembly according to embodiments of the present invention.

FIG. 21 is a partial view of components of the system shown in FIG. 16A.

FIGS. 22A and 22B are side views of the system shown in FIG. 16A illustrating the surgical frame mount assembly in different orientations provided by the angling plate according to embodiments of the present invention.

FIG. 22C is a side perspective view of the system shown in FIG. 16A without a patient held in the system according to embodiments of the present invention.

FIG. 22D is a partial exploded view of the system shown in FIG. 22C.

FIG. 23A is an enlarged, partial top perspective view of the surgical frame mount assembly shown in FIG. 16A.

FIG. 23B is a side perspective view of a surgical frame mount assembly of the system shown in FIG. 22C according to embodiments of the present invention.

FIG. 23C is a partial exploded view of the assembly shown in FIG. 23B.

FIG. 23D is a side perspective view of the surgical frame mount assembly shown in FIG. 23B with the coupling arm and orientation plate partially exploded and aligned relative thereto according to embodiments of the present invention.

FIG. 24A is a side view of a support arm assembly of the frame mount assembly shown in FIG. 23B according to embodiments of the present invention.

FIG. 24B is a partial exploded view of the support arm assembly shown in FIG. 24A.

FIGS. 25A-25C illustrate the system of FIG. 1A with two support arms for a bilateral procedure according to embodiments of the present invention.

### DETAILED DESCRIPTION OF EMBODIMENTS OF THE INVENTION

The present invention now is described more fully hereinafter with reference to the accompanying drawings, in which some embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

Like numbers refer to like elements throughout. In the figures, the thickness of certain lines, layers, components, elements or features may be exaggerated for clarity.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when an element is referred to as being “on”, “attached” to, “connected” to, “coupled” with, “contacting”, “supported by” etc., another element, it can be directly on, attached to, connected to, coupled with or contacting the other element or intervening elements may also be present (e.g., indirectly supported, attached, coupled, contacting, connected, coupled, etc. . . . ). In contrast, when an element is referred to as being, for example, “directly on”, “directly attached” to, “directly connected” to, “directly coupled” with, “directly supported by” or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is inverted, elements described as “under” or “beneath” other elements or features would then be oriented “over” the other elements or features. Thus, the exemplary term “under” can encompass both an orientation of “over” and “under.” The device may be otherwise oriented (rotated 90 degrees or at other orientations) and the spatially relative descriptors used herein interpreted accordingly. Similarly, the terms “upwardly,” “downwardly,” “vertical,” “horizontal” and the like are used herein for the purpose of explanation only unless specifically indicated otherwise.

The term “about” with respect to a number indicates that the value may vary between +/−20%.

The term “scanner bed” is used interchangeably with “table” and refers to a patient support surface or frame thereof (which is typically relatively rigid) that, in operative position, resides in a scanner, such as a CT or MRI scanner. For MRI use, the scanner bed resides in a region of a homogeneous high magnetic field associated with a Magnetic Resonance Imaging (MM) scanner during active image signal acquisition. The scanner bed can typically translate in a longitudinal direction to position the patient in the homogeneous magnetic field region of the magnet. MRI scanners are well known to those of skill in the art and include, for example, the SIGNA 1.5 T/3.0 T from GE Healthcare: the ACHEIVA 1.5 T/3.0 T and the INTEGRA 1.5 T from Philips Medical System; and the MAGNETOM Avanto, the MAGNETOM Espree, the MAGNETOM Symphony, and the MAGNETOM Trio, from Siemens Medical. CT Scanners are also well known in the art.

The term “MRI-compatible” means that a device is safe for use in an MRI environment that can operate as intended in an MM environment and not introduce artifacts into MM images. As such, if residing within the high-magnetic field region of the magnet, the MRI-compatible device is typically made of a non-ferromagnetic material(s) suitable to reside and/or operate in a high magnetic field environment. The term “high magnetic field” refers to magnetic fields above 0.5 T, typically between 1.5 T to 10 T.

The term “tool” refers to devices that facilitate medical procedures.

Embodiments of the invention are particularly suitable for veterinarian or human therapeutic or diagnostic use but may be used for research or other purposes.

The term “sterile” and derivatives thereof means that the component meets regulatory clinical cleanliness standards for medical procedures.

The term “fiducial marker” refers to a marker that can be electronically identified using image recognition and/or electronic interrogation, typically interrogation of CT or MRI image data. The fiducial marker can be provided in any suitable manner, such as, but not limited to, a geometric shape, a component on or in the device, optical or electrical tracking coils, a coating or fluid-filled component or feature (or combinations of different types of fiducial markers) that makes the fiducial marker(s) Mill-visible or CT visible with sufficient signal intensity (brightness) for identifying location and/or orientation information for the device and/or components thereof in space.

The term “grid” refers to a pattern of crossed lines or shapes used as a reference for locating points or small spaces, e.g., a series of rows and intersecting columns, such as horizontal rows and vertical columns (but orientations other than vertical and horizontal can also be used). The grid can include at least one fiducial marker. The grid can include associated visual indicia such as alphabetical markings (e.g., A-Z and the like) for rows and numbers for columns (e.g., 1-10) or the reverse. Other marking indicia may also be used. The grid can be provided as a flexible patch that can be releasably attached to the skin of a patient. For additional description of suitable grid devices, see co-assigned U.S. patent application Ser. No. 12/236,621 (U.S. Pat. No. 8,195,272), the contents of which are hereby incorporated by reference as if recited in full herein.

Embodiments of the present invention can be configured to carry out or facilitate CT or MM guided procedures, including, for example diagnostic and interventional procedures such as to guide and/or place interventional devices to any desired internal region of the body or object, including deep brain sites for neurosurgeries or other target intrabody locations for other procedures. The object can be any object and may be particularly suitable for animal and/or human subjects. For example, the system and/or devices thereof can be used for gene, e.g., antibody, and/or stem-cell based therapy delivery or other therapy delivery to intrabody targets in the brain, heart, lungs, liver, kidney, ovary, stomach, intestine, colon, spine or to other locations. In addition, embodiments of the systems can be used to treat cancer sites. In some embodiments, the systems can be used to ablate tissue and/or delivery pharmacologic material in the brain, heart or other locations. In some embodiments, it is contemplated that the systems can be configured to treat AFIB, deliver stem cells or other cardio-rebuilding cells or products into cardiac tissue, such as a heart wall, via a minimally invasive MRI guided procedure while the heart is beating (i.e., not requiring a non-beating heart with the patient on a heart-lung machine).

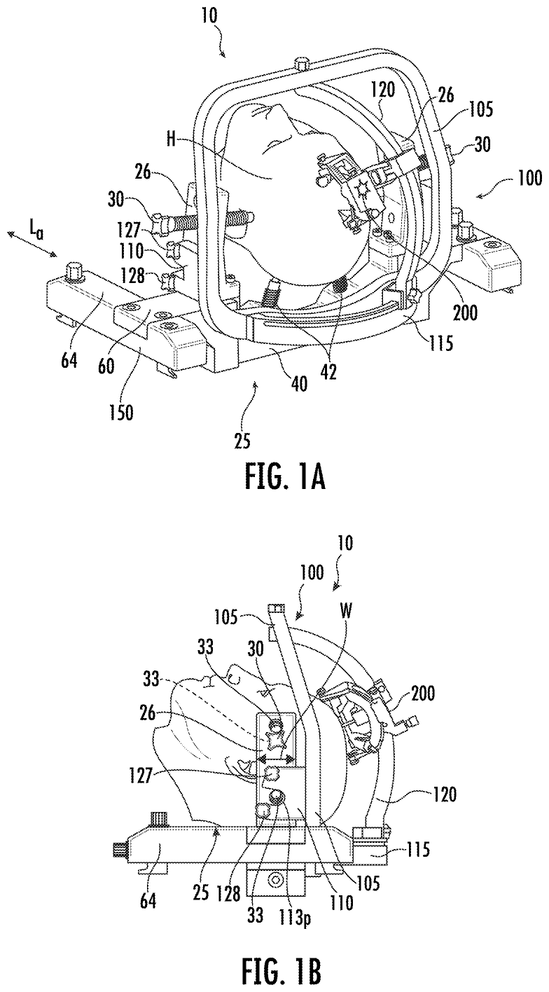

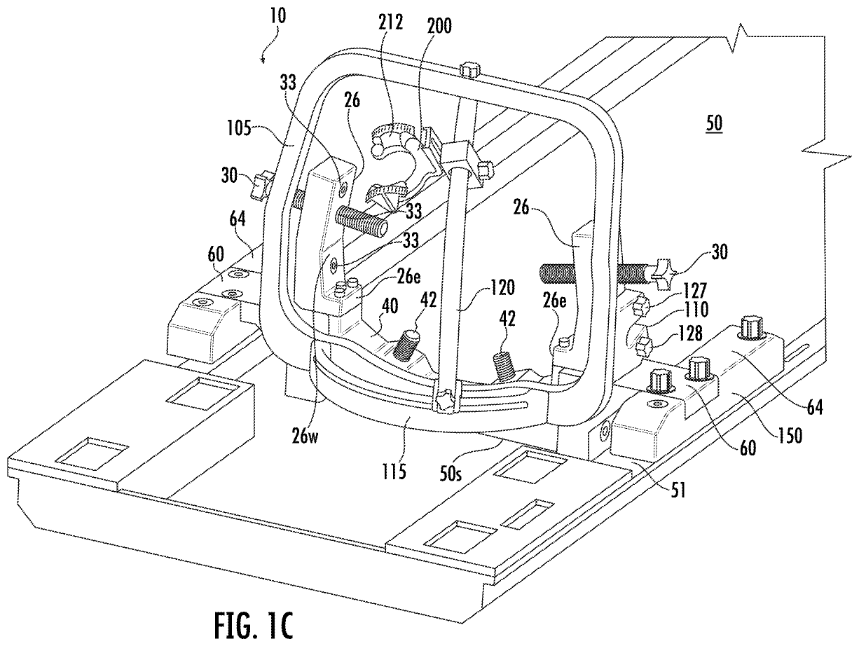

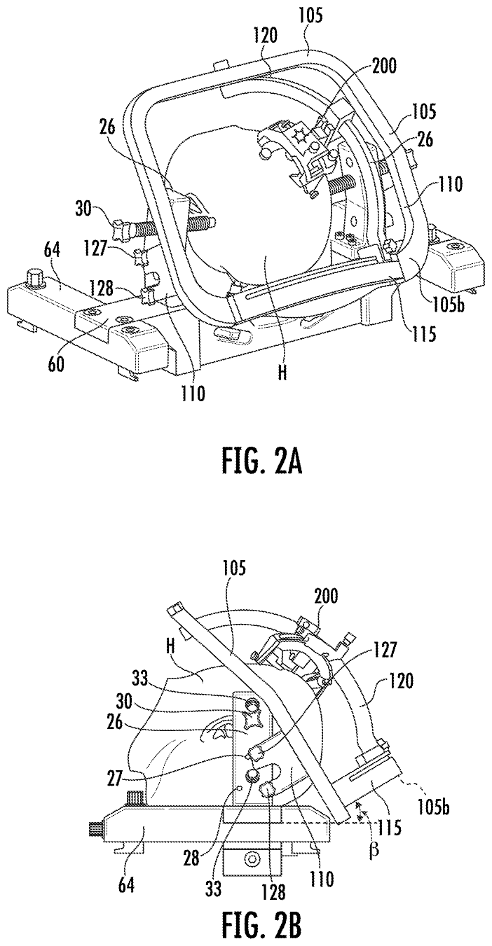

Referring now FIGS. 1A, 1B, 1C, 2A, and 2B, an example surgical tool support system 10 is shown. The surgical tool support system 10 comprises a head fixation assembly 25 and a surgical frame mount assembly 100. The system 10 can include support members 26 attached to respective table mounts 150. The support members 26 can couple to or form part of the head fixation assembly 25. The support members 26 can extend upwardly from the table mounts 150. The support members 26 can also be referred to as side (support) members 26 and can be provided as a right side and left side (support) member 26 that are spaced laterally apart a sufficient distance to allow a patient head H to be received therebetween.

The head fixation assembly 25 can be configured to receive a plurality of skull fixation members 30. The side members 26 can comprise vertically spaced apart apertures 33 that are sized and configured to receive fixation members, which may be respective head fixation members 30.

Referring to FIG. 1C, for example, the head fixation assembly 25 can also comprise a base member 40. The base member 40 can be configured to reside on a patient support surface 50s. The base member 40 can be configured to provide additional head fixation members 42 that extend upwardly. The base member 40 can abut and couple to an inner end 26e of the side (support) members 26.

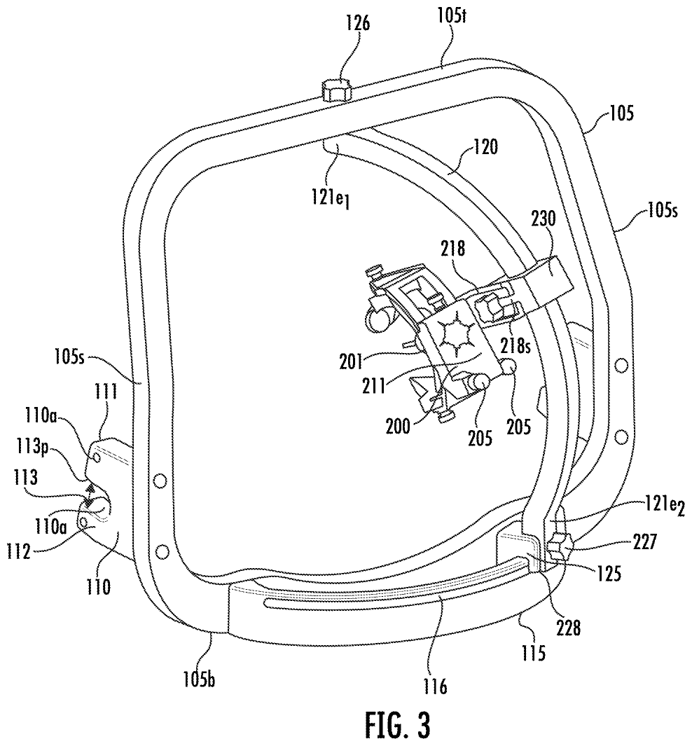

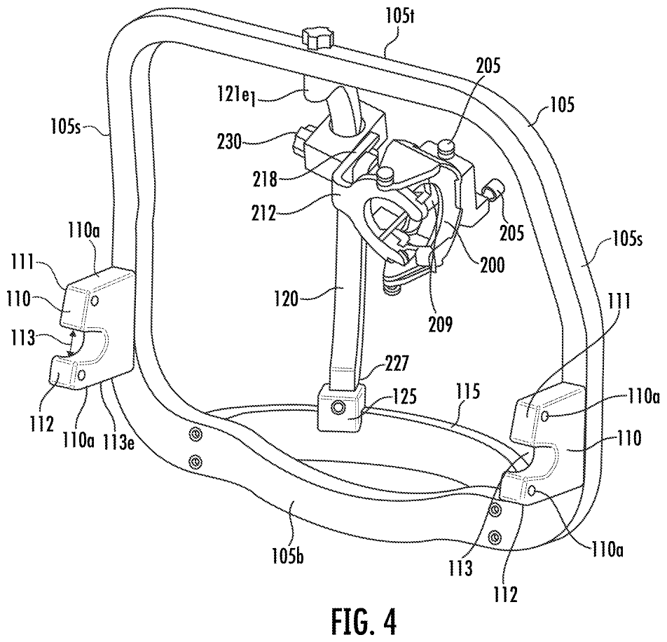

Referring also to FIGS. 3 and 4, the surgical frame mount assembly 100 comprises a frame member 105, a base 115 and at least one support arm 120 coupled to the frame member 105 and the base 115. As shown, the surgical frame mount assembly 100 also includes first and second coupling arms 110 extending longitudinally off respective right and left sides 105s of the frame member 105. Each coupling arm 110 can attach to a support/side member 26.

The coupling arms 110 can have an upper arm segment 111 and a lower arm segment 112 separated by a gap space 113. The gap space 113 can be defined by a curvilinear perimeter opening between the upper and lower arm segments 111, 112. The curvilinear perimeter 113p can provide access to a fixation aperture 33 in a respective side member 26 as shown in FIG. 1B.

The coupling arms 110 can be interchangeably referred to as “angle plates” as they can be oriented at different angles with respect to the patient and/or side members 26 for different lockable positions.

As shown in FIGS. 1A, 1B, 2A, 2B, 3 and 4, for example, a surgical tool 200 is held by the support arm 120. The support tool 200 can comprise a trajectory guide 201.

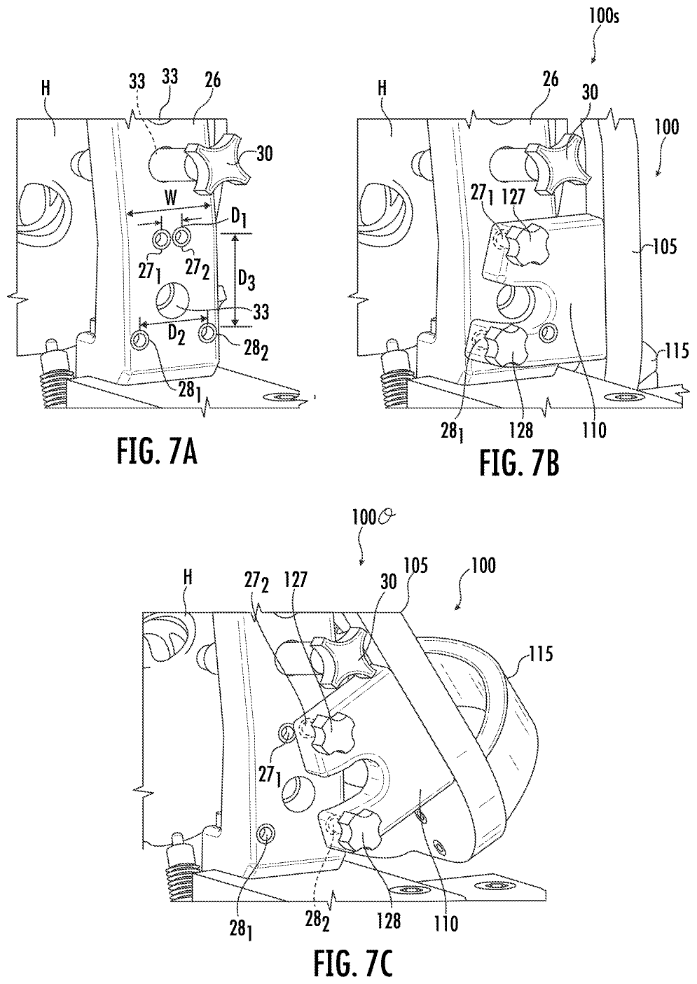

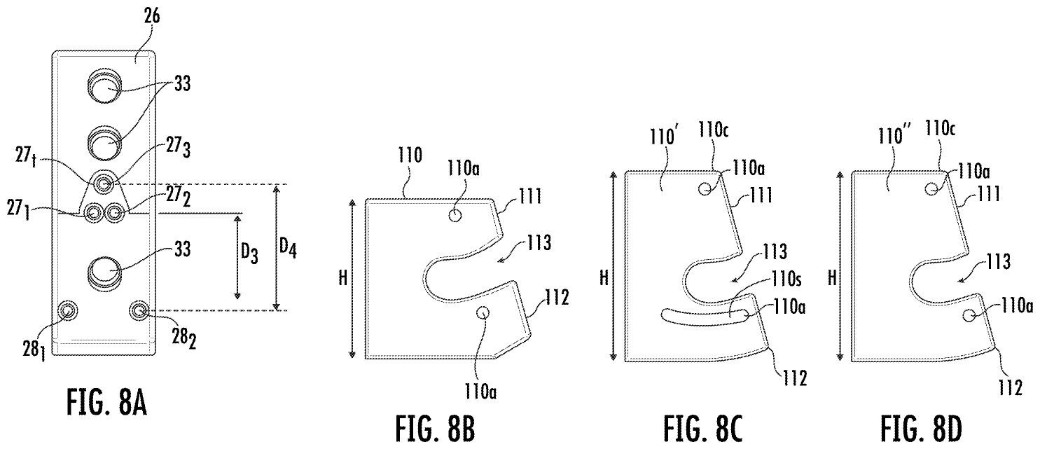

Referring to FIGS. 7B and 8B-8D, the coupling arms 110, 110′, 110″ can be configured so that the upper arm segment 111 and the lower arm segment 112 each comprises through apertures 110a sized and configured to receive respective attachment members 127, 128. The aperture 110a of the lower arm segment 112 can comprise an arcuate slot 110s (FIG. 8C). Attachment members 127, 128 can extend through the apertures 110a of each coupling arms 110, 110′, 110″ to couple to a corresponding side member 26 of the head fixation assembly 25.

Referring to FIGS. 7A and 8A, the side member(s) 26 of the head fixation assembly 25 can comprise at least one first attachment aperture 27 residing above a second at least one second attachment aperture 28 that receive the attachment members 127, 128 to couple respective coupling arms 110, 110′, 110″. The at least one first attachment aperture 27 may be provided as a single aperture or provided as a plurality of spaced apart first attachment apertures. The at least one second attachment aperture 28 can be provided as a single aperture or a plurality of laterally spaced apart attachment apertures, shown as laterally spaced apart apertures 28₁, 28₂ in FIGS. 7A and 8A.

FIG. 7A illustrates that the at least one first attachment aperture 27 can be provided as first and second laterally spaced apart apertures 27₁, 27₂. FIG. 8A illustrates that the first attachment aperture 27 can be provided as a plurality of spaced apart apertures 27₁, 27₂, 27₃, with one provided as a top aperture 27t, positioned above the other two.

FIGS. 8B-8C illustrate that the coupling arms 110, 110′, 110″ can be provided with different heights H. The coupling arms 110′, 110″ of FIGS. 8C and 8D, respectively, can be configured with heights H that position the aperture 110a adjacent an inner facing corner 110c and alignable with a top attachment aperture 27t (e.g., pivot hole) of the side member 26 (FIG. 8A).

While FIG. 8A illustrates the side member 26 with three first attachment apertures 27, a single one may be provided for some embodiments. In some embodiments, only two laterally spaced apart apertures 27₁, 27₂ can be provided, as shown in FIG. 7A.

As shown in FIG. 8A, a plurality of head fixation apertures 33 of larger diameter than the coupling arm 110, 110′, 110″ attachment apertures 27, 28 and can reside above the first aperture(s) 27. At least one head fixation aperture 33 can reside between the first and second attachment apertures 27, 28.

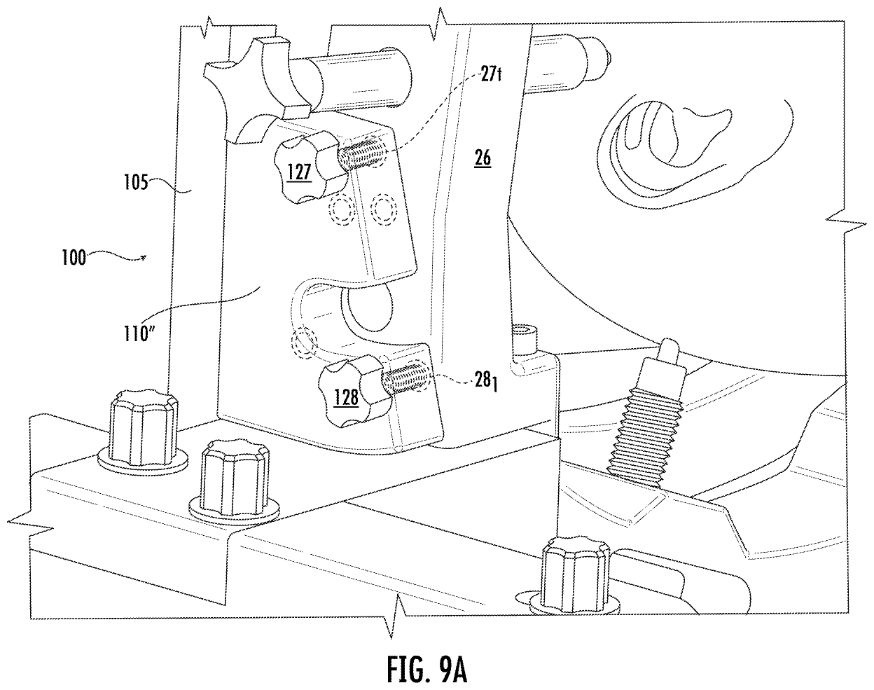

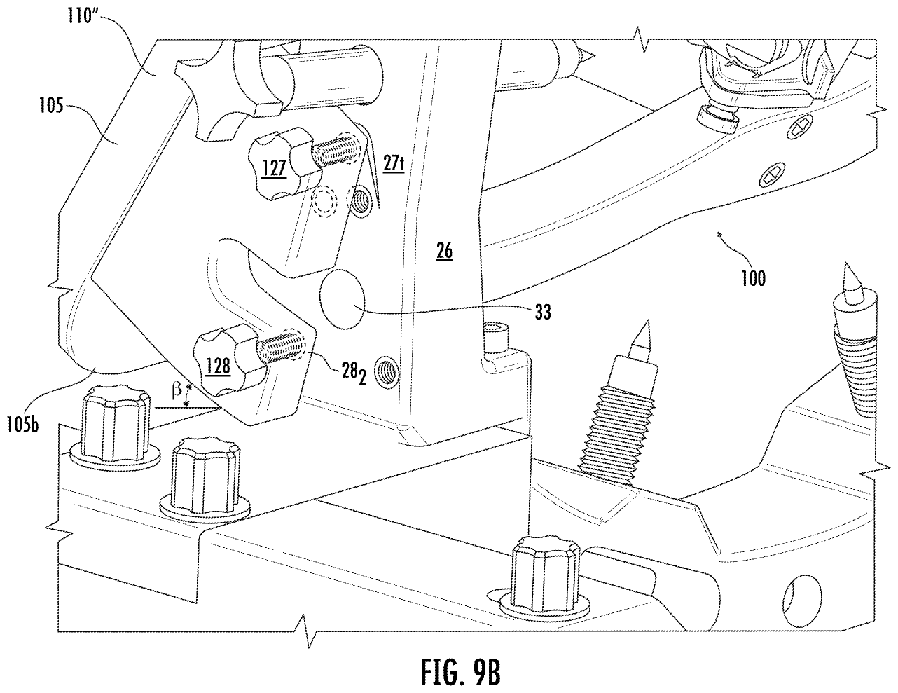

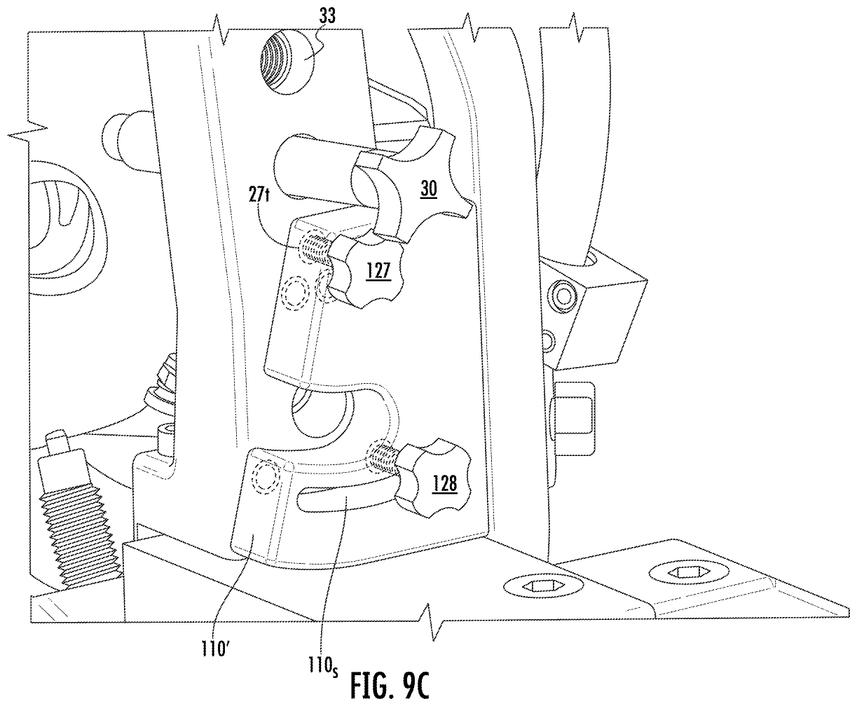

Configuring the side members 26 with three first attachment apertures 27₁, 27₂, 27₃, allows for interchangeable use with different coupling arms 110, such as coupling arms 110′, 110″ shown in FIGS. 8C and 8D with a greater height than shorter coupling arm 110 shown in FIG. 8B (and FIGS. 7B, 7C).

Where more than one first attachment aperture 27 is provided, the coupling arm 110, 110′, 110″ can couple, at any one time, to a single first aperture 27 and to either (i) a single one of the second plurality of apertures 28 at any one time (FIGS. 7B, 7C, 9A, 9B) or (ii) a desired slot location 110s (FIG. 9C). The slot 110s may allow “on the fly” angulation adjustment rather than a choice between predefined angulation positions.

The attachment members 127, 128 (optionally thumb screws) for the coupling arms 110, 110′, 110″ of the surgical frame mount assembly 100 can be used to couple a respective coupling arm 110, 110′, 110″ to the head fixation assembly 25 selectively using a first attachment aperture 27 and a second attachment aperture 28 for each of the side members 26 to lock into either a first orientation for a supine procedure (FIGS. 1A, 1B) or a second orientation for an occipital procedure (FIGS. 2A, 2B).

Referring to FIG. 2B, the base 115 of the frame mount assembly 100 can have a second orientation that is at an angle “β” from horizontal that is in a range of 15-60 degrees, more typically a range of 30-45 degrees, in some embodiments. In contrast, as shown in FIG. 1B, the base 115 and bottom 105b of the frame member 105 can be substantially horizontal (e.g., within +/−5 degrees) in the first orientation.

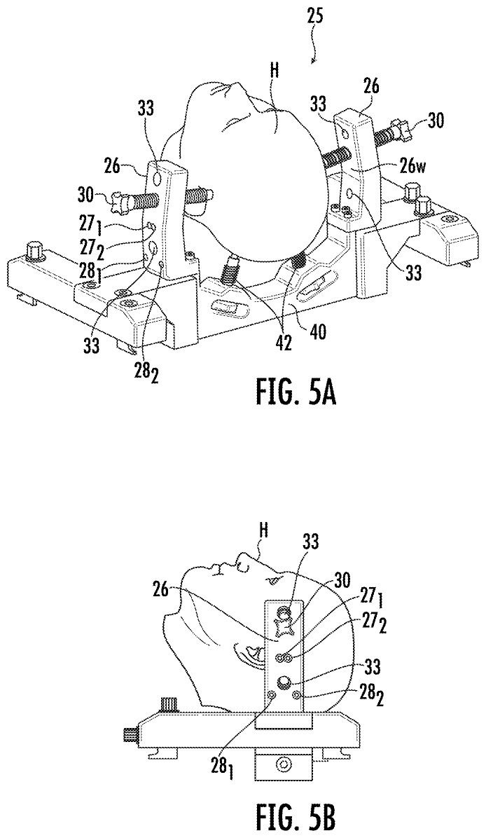

As shown in FIGS. 1C and 5A, the attachment apertures 27, 28 for the attachment members 127, 128 can be “blind” apertures so that the attachment members 127, 128 do not extend through and out of an inner surface of the wall 26w of the side members 26 when coupled to the side members 26.

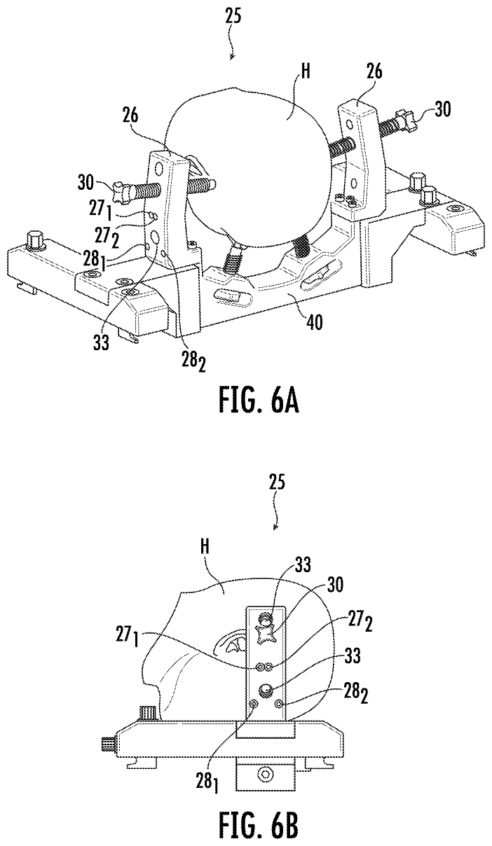

Referring to FIGS. 5A, 5B, 6A, 6B, and 7A, for example, the first and second apertures 27, 28 for the attachment members 127, 128, respectively, can have a smaller diameter than the head fixation apertures 33. Although shown as having a common diameter, the first and second attachment apertures 27, 28 can have different sizes.

Referring to FIG. 7A, the first attachment aperture 27 comprises a first aperture 27₁ and a second aperture 27₂ that are laterally spaced apart a distance D₁ (center hole to center hole) in a width dimension W associated with a length dimension and/or longitudinal axis La of a patient support table. The second plurality of apertures 28 can include a first aperture 28₁ and a second aperture 28₂ that are also laterally spaced apart a (center hole to center hole) distance D₂ in the width dimension W associated with a length dimension and/or longitudinal axis La of a patient support table. D₂>D₁. D₂ can be 2×-5× greater than D₁, in some embodiments. In some embodiments, D₁ is in a range of about 0.25 to about 0.50 inches, such as about 0.42 inches. In some embodiments, D₂ is in a range of about 1.0 inches to about 1.75 inches, such as about 1.45 inches.

Referring to FIG. 7A, the first attachment aperture 27 can reside a distance D₃ above the second attachment aperture(s) 28. D₃ (center hole to center hole) can be in a range of about 2-3 inches, typically about 2 inches.

Referring to FIG. 8A, where a top aperture 27t is provided (also labeled as callout 27₃ where provided as a set of first attachment apertures 27) and which can also be described as a pivot hole for the coupling arm 110), the top aperture 27t can reside a distance D₄ above the second attachment apertures 28 (center hole to center hole). D₄ can be in a range of about 2.5-3.5 inches, typically about 3 inches.

The first plurality of apertures 27 can be laterally aligned (reside at a common horizontal position). The second plurality of apertures 28 can be laterally aligned (reside at a common horizontal position) under the first plurality of apertures 27. The first plurality of apertures 27 can reside a distance “D₃” above the second plurality of apertures. In some embodiments, D₃ is in a range of about 1 inch to about 3 inches, such as about 2.5 inches. The distances can allow the proper angulation for a wide range of motion of the frame mount 100 assembly relative to the head fixation assembly 25.

As shown in FIG. 1C, the surgical support system 10 can comprise a table mount 150 that can be attached to a scanner table or bed 50 (e.g., a patient support surface) of an Mill, CT or other imaging scanner. In the embodiment shown, the head fixation assembly 25 comprises laterally extending members 60 that have opposing inner and outer end portions. The inner end portions are coupled to the side members 26 while the outer end portions are attached to the longitudinally extending coupling members 64 that are attached to sides 51 of the scanner bed 50.

In some embodiments, the vertically spaced apart apertures 33 comprise at least two apertures 33, shown as three, typically provided in a range of 2-6 apertures at different height positions to accommodate different size heads of respective patients and/or supine and occipital positions during a surgical procedure.

FIGS. 5A, 5B and 7A illustrate an example head fixation assembly 25 without the surgical frame mount assembly 100.

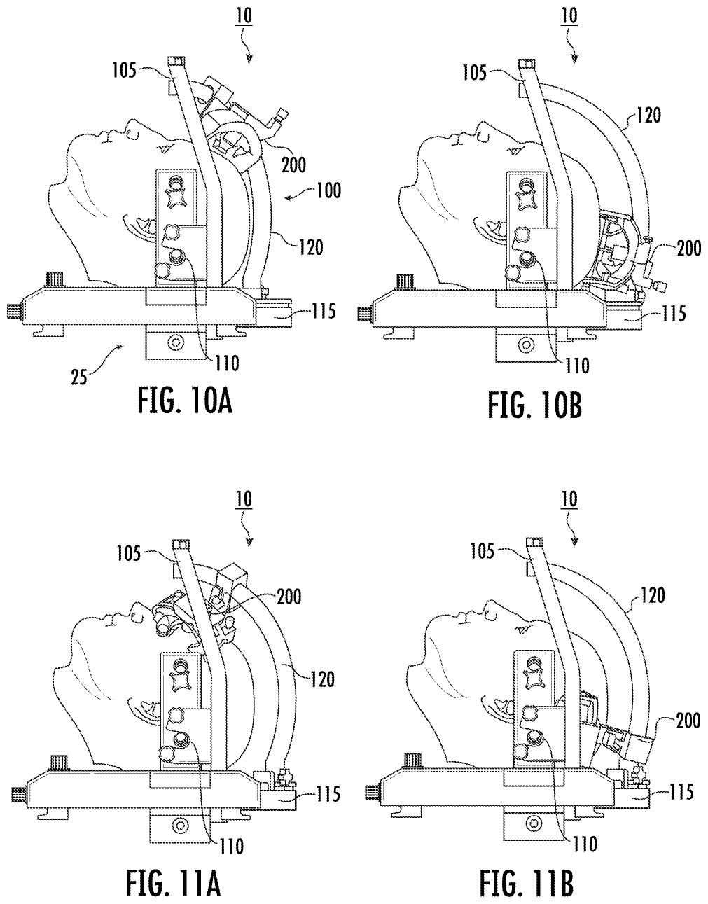

FIG. 7B illustrates the coupling arm 110 coupled to the side member 26 using a first set of apertures 27₁, 28ₗ with the surgical frame mount assembly 100 in a first locked orientation/position for a supine procedure and placing the base 115 and top 105t of the frame member 105 in a substantially horizontal position (within +/−10% of horizontal) and with the top 105t above a head H of the patient (FIGS. 10A, 10B, 11A and 11B).

FIG. 7C illustrates the coupling arm 110 coupled to the side member 26 using a different set of apertures 27₂, 28₂ with the surgical frame mount assembly 100 in a second locked orientation/position for an occipital procedure.

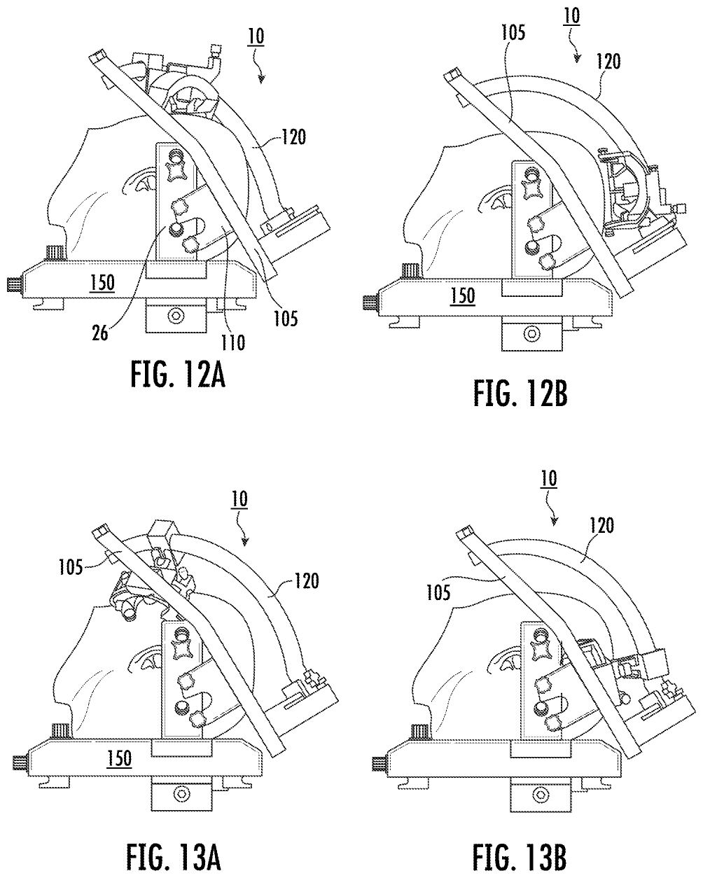

The base 115, the support arm 120 and the frame member 105 can move and/or pivot as a unit to lock into the second position which moves the base 115 upward and the top 105t of the frame member 105 forward (FIG. 12A, 12B, 13A, 13B) relative to the first position and with the top 105t above a head H of the patient.

FIGS. 9A and 9B illustrate the coupling arm 110″ of FIG. 8D attached to the side member 26 of the head fixation assembly 25 using the top attachment aperture 27t. FIG. 9A shows the coupling arm 110″ also attached to a first one 28₁ of the second attachment apertures 28 while FIG. 9B shows the coupling arm 110″ also attached to a second one 28₂ of the second attachment apertures whereby the frame assembly 100 can pivot as a unit to an angle θ from horizontal as discussed above with respect to FIG. 2B.

FIG. 9C illustrates the coupling arm 110′ of FIG. 8C attached to the side member 26 of the head fixation assembly 25 using the top attachment aperture 27t (e.g., pivot hole) and attachment member 127. FIG. 9C shows the coupling arm 110′ also attached to the slot 110s and one attachment aperture 28, shown as attachment aperture 28₂ using attachment member 128.

FIGS. 10A and 10B illustrate respective example medial maxima (max) and minima (min) positions of the surgical tool 200 held by the support arm 120 when the patient and system 10 are in the supine position/orientation using coupling arm 110, 110′, 110″. FIGS. 11A and 11B illustrate respective lateral max and min positions allowed by the supine orientation.

FIGS. 12A and 12B illustrate respective medial max and min positions of the surgical tool 200 held by the support arm 120 when the patient and system 10 are in the occipital position/orientation. FIGS. 13A and 13B illustrate respective lateral max and min positions allowed by the occipital orientation.

The surgical tool 200 supported by the support arm 120 can comprise a trajectory guide 201 with or without a targeting cannula for allowing components such as catheters, needles, leads with electrodes, drill bits, fluid delivery cannulae, or other devices to be inserted into a patient's body along a desired intrabody path through the guide. The tool 200 can reside on or above a patient. The tool 200 can reside against/on an outer surface or skull of a patient for the surgical procedure.

In some embodiments, the tool 200 may be configured to be supported by the system 10 without requiring attachment to a skull of a patient which may be particularly suitable for use with some patients such as pediatric patients or patients with thin skulls or other skull abnormalities.

Referring to FIGS. 3 and 4, the surgical tool 200 can comprise a trajectory guide assembly 201 that can include a support column 209 coupled to a platform 211 for X-Y adjustment, and actuators 205 that can adjust the pitch, roll and X-Y adjustments of the trajectory. The trajectory guide 201 can include a base 212 that can be semi-circular and can have an upwardly extending leg 218 with a slot 218s that couples to the arm attachment member 230. A lock screw, clamp, pin or other locking member 230m can be used to lock the arm attachment member 230 a desired location on the support arm 120.

The leg 218 can allow the trajectory guide 201 to be adjusted up and down relative to the support arm 120. The trajectory guide 201 can include at least one fiducial marker. For additional discussion of suitable trajectory guides, see, U.S. application Ser. No. 12/134,412, co-assigned U.S. patent application Ser. Nos. 12/236,950, and 14/515,105, the contents of which are hereby incorporated by reference as if recited in full herein.

The head fixation assembly 25 can cooperate with RF coils to obtain MRI signals. For additional description of suitable head fixation frames according to some embodiments, see, e.g., U.S. Pat. No. 8,548,569, the contents of which are hereby incorporated by reference as if recited in full herein.

Referring again to FIGS. 3 and 4, the frame member 105 can have a closed perimeter with a top 105t, a bottom 105b and sides 105s that couple the top 105t to the bottom 105b. The base 115 can be coupled to the bottom 105b. The support arm 120 can be coupled to the base 115 and the top 105t of the frame member 105.

The support arm 120 of the surgical frame mount assembly 100 can include opposing upper and lower end portions 121e₁, 121e₂. The upper end portion 121e₁ can be coupled to an upper attachment member 126 to attach to the top 105t of the frame member 105. The lower end portion 121e₂ can be coupled to a lower attachment member 227 that attaches to the base 115. The lower end portion 121e₂ can include or be coupled to a block member 125 that can form part of the attachment member 227 and that slidably couples to the base 115. The block member 125 and/or the lower end of the support arm 121e₂ can include a ridge 228 that projects inwardly and engages a slot 116 in the base 115 to pivot the arm 120 about the upper attachment member 126 laterally along a length of the base 115 to position the arm in different positions relative to the sides 105s of the frame member 105.

The support arm 120 can have a length associated with a height dimension that is between about 6-12 inches, in some embodiments. In some particular embodiments, the support arm 120 can have a length of between 9-12 inches, including about 9 inches, about 10 inches, about 11 inches and about 12 inches. The arm 120 can be curvilinear, shown as having an arcuate body.

It is contemplated that one can use a single support arm 120 or, in some embodiments, a plurality of support arms 120 for neurological, e.g., brain surgeries or other target anatomical locations using an appropriate arm (arc) design.

In some embodiments, the support arm 120 can have an arcuate segment with a radius of curvature that is between 100 mm and 150 mm, typically between about 110 mm and 140 mm, such as about 110 mm, about 120 mm, about 130 mm, and about 140 mm.

The system 10 can be sized and configured to fit within the bounds of a bore of a magnet (for closed bore systems) and can translate in and out of the magnet bore with the patient and scanner bed 50 (FIG. 1C) and remain in a fixed position relative to the patient.

Components of the system 10 can be formed from any suitable material, typically a light-weight relatively rigid polymeric material, such as, for example, fiberglass, ceramics, fiber reinforced resins, PEEK, ABS, polycarbonate, KEVLAR, and/or Garolite. However, non-ferromagnetic metals or other materials may also be used, particularly when used for non-MRI surgical navigation systems.

The systems 10 may be particularly suitable for use in MM-guided procedures where the procedure is carried out in an MM scanner or MM interventional suite, e.g., deep brain procedures, spinal procedures, cardiac procedures, including but not limited to, cardiac EP procedures where heat or cryogenic ablation is used, as well as intrabody biopsies or treatment of any target organ or tissue, including breast, liver, thyroid, lung, kidney, ovarian, cervical, prostate, urethra, colon, intestine, stomach, and the like. The devices be suitable for MRI-guided procedures that deliver therapeutic agents, such as drugs, antigen, antibody and/or gene therapies, stem cells and the like. However, use in non-MRI image guided systems are also contemplated.

The system 10 (or appropriate components of one or both, depending on use) can be sterilized and may optionally be single-use disposable or portions thereof may be single-use disposable. The devices can be “universal” in that one or both can be used interchangeably with different MM scanner systems from different scanner manufacturers. Alternatively, the systems 10 may have different configurations of the attachment members used to attach to different Scanner beds, e.g., they may be scanner type or scanner manufacturer specific.

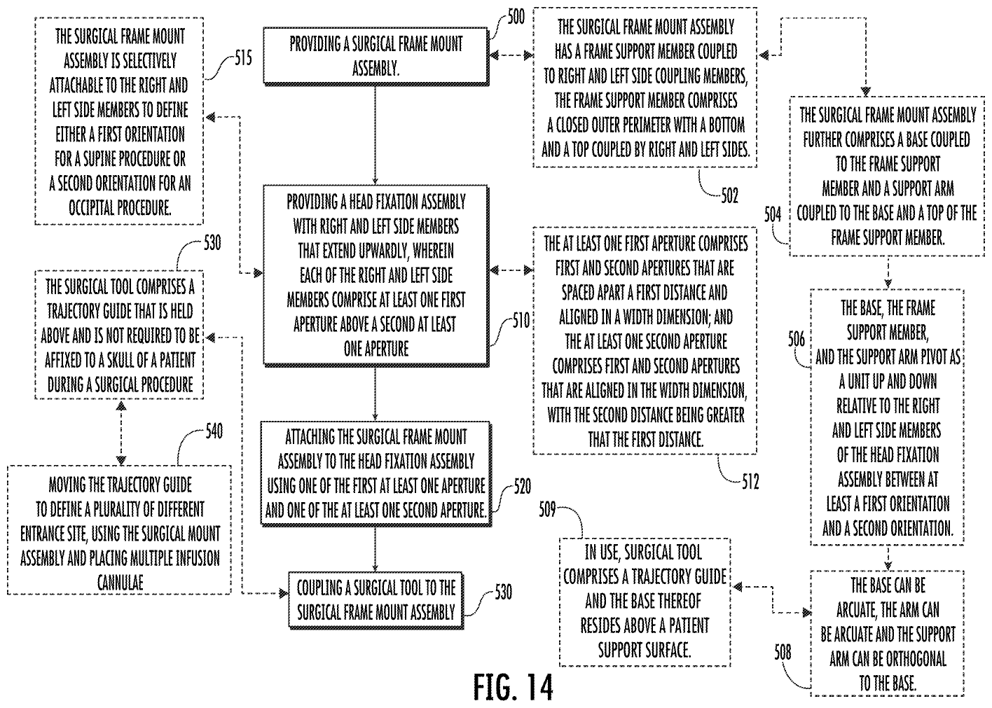

FIG. 14 illustrates example operations that can be used for a surgical procedure. A surgical frame mount assembly is provided (block 500). A head fixation assembly with right and left side members that extend upwardly are provided, each of the right and left side members comprise at least one first aperture above at least one second aperture (block 510). The surgical frame mount assembly is attached to the head fixation assembly using one of the at least one first aperture and one of the at least one second aperture (block 520). A surgical tool is coupled to the surgical frame mount assembly (block 530).

The surgical frame mount assembly has a frame member coupled to right and left side coupling arms, the frame member comprises a closed outer perimeter with a bottom and a top coupled by right and left sides (block 502).

The at least one first aperture comprises first and second apertures that are spaced apart a first distance and aligned in a width dimension; and the at least one second aperture comprises first and second apertures that are spaced apart a second distance and aligned in the width dimension, with the second distance being greater than the first distance (block 512).

The surgical frame mount assembly further comprises a base coupled to the frame member and a support arm coupled to the base and a top of the frame member (block 504).

The base, the frame member, and the support arm are pivotable as a unit up and down relative to the right and left side members of the head fixation assembly between at least a first orientation (first locked position) and a second orientation (second locked position) (block 506).

The base can be arcuate, the support arm can be arcuate and the support arm can be orthogonal to the base (block 508).

In use, the trajectory guide and the base reside above a patient support surface (block 509).

The surgical frame mount assembly can be selectively attached/attachable to the right and left side members to define either a first orientation for a supine procedure or a second orientation for an occipital procedure (block 515).

The surgical tool can comprise a trajectory guide that is not required to be affixed to a head of a patient during a surgical procedure (block 530).

The trajectory guide can be moved to different selected sites during an interventional procedure while coupled to the frame mount assembly, and the surgical tool can be used to place a plurality of infusion cannulae in the head of the patient at respective different selected sites during the interventional procedure, such as an infusion treatment (block 540).

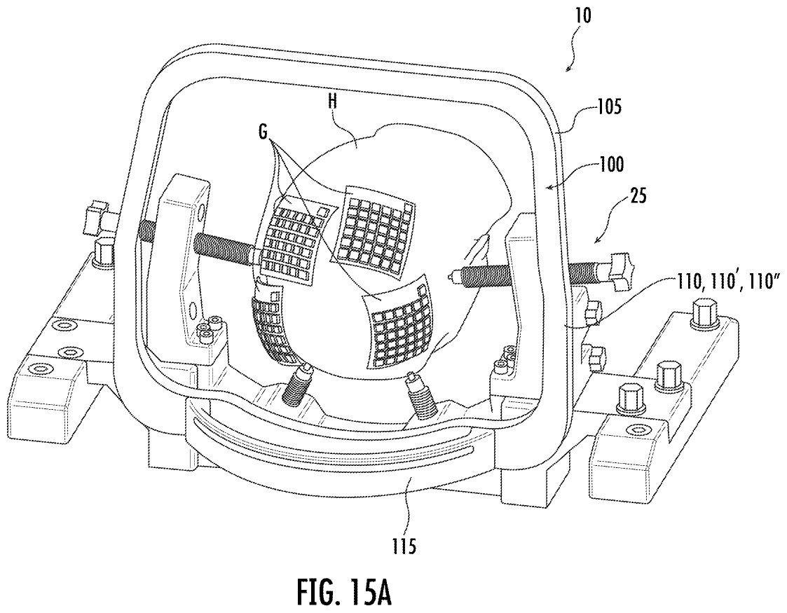

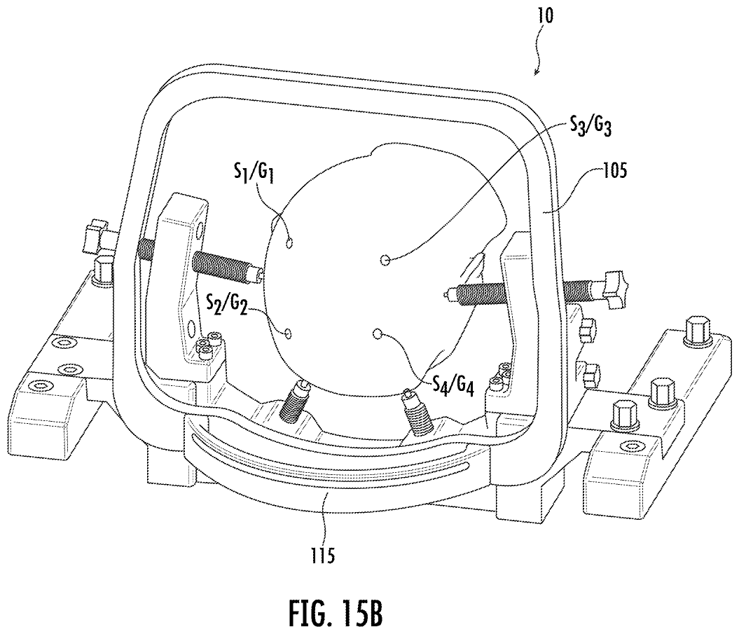

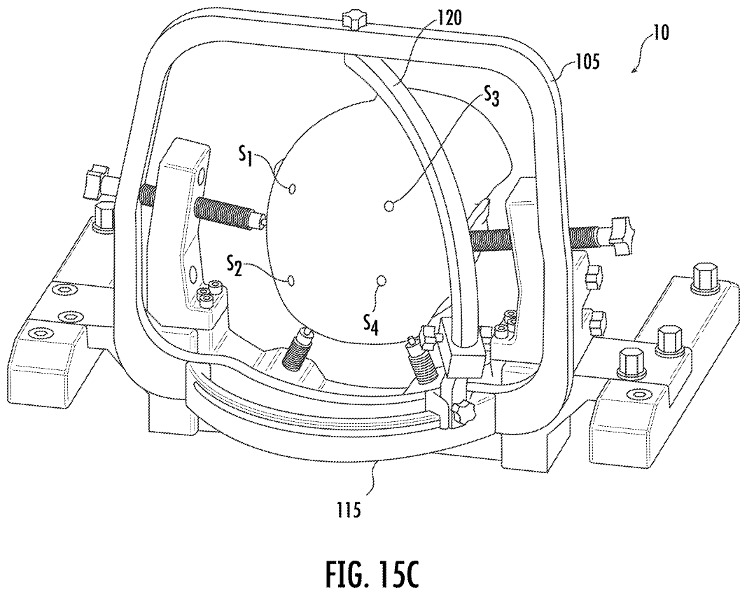

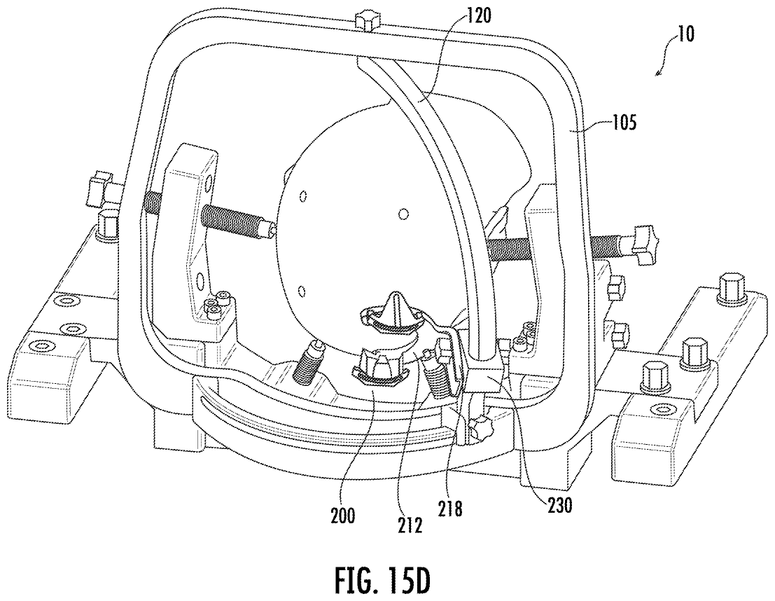

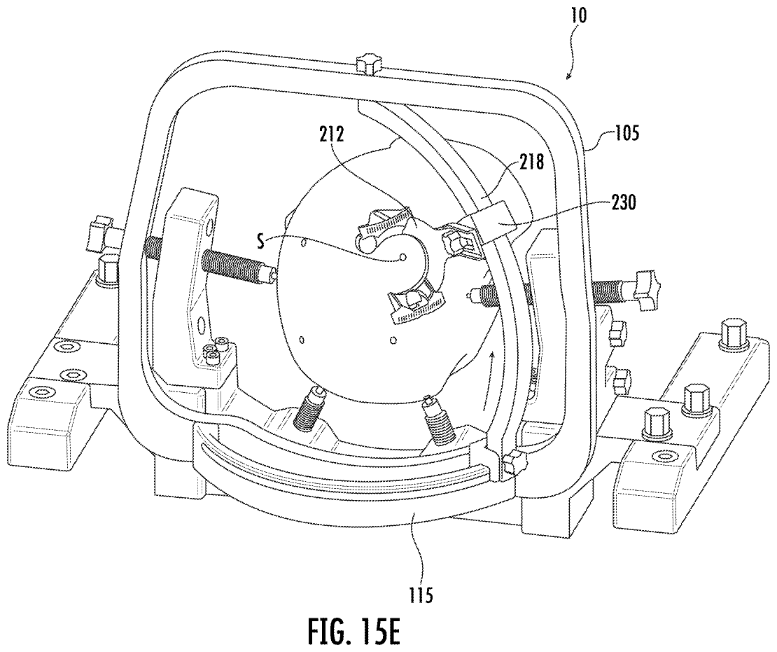

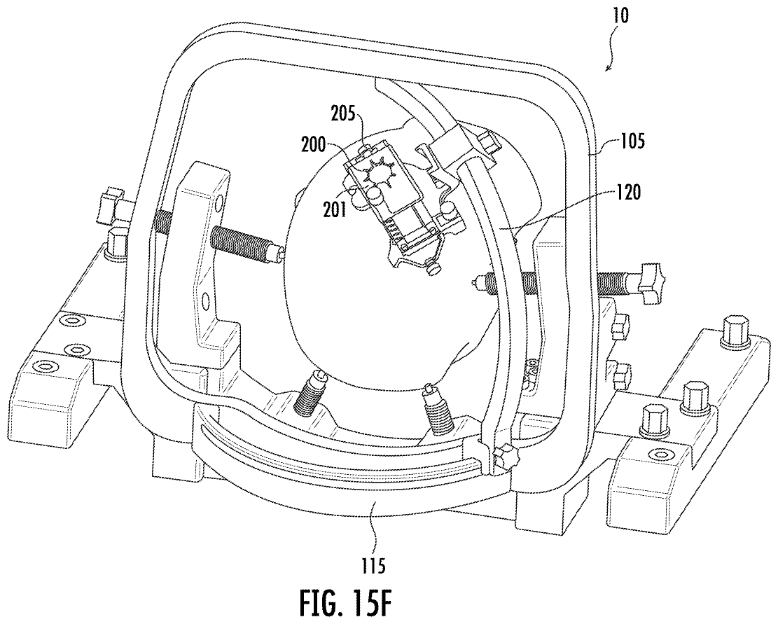

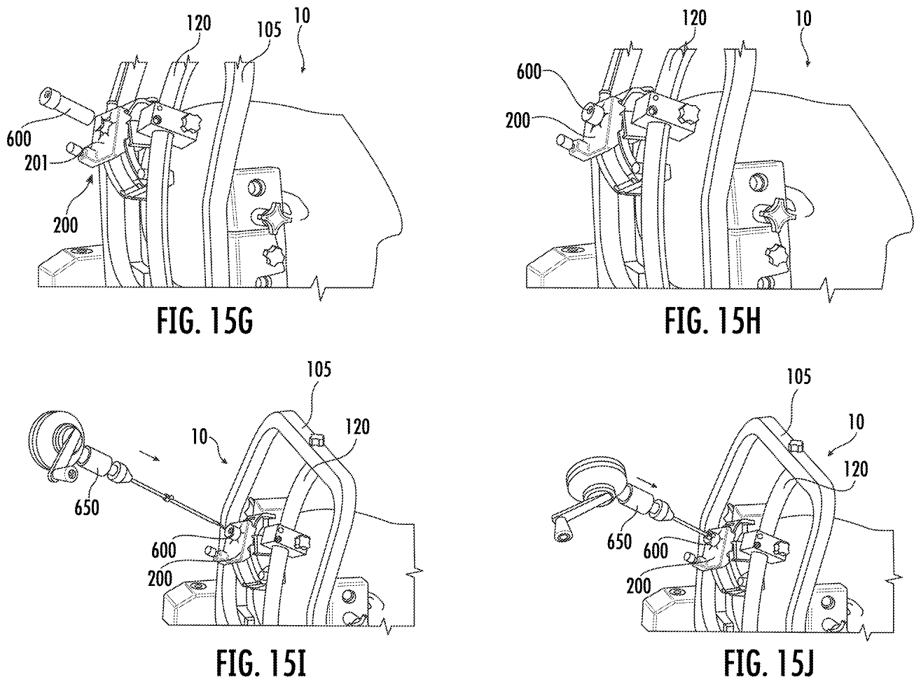

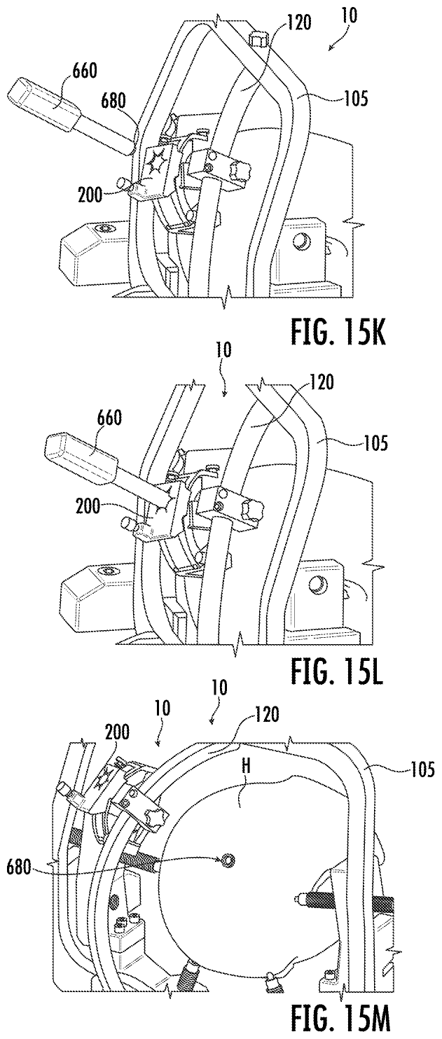

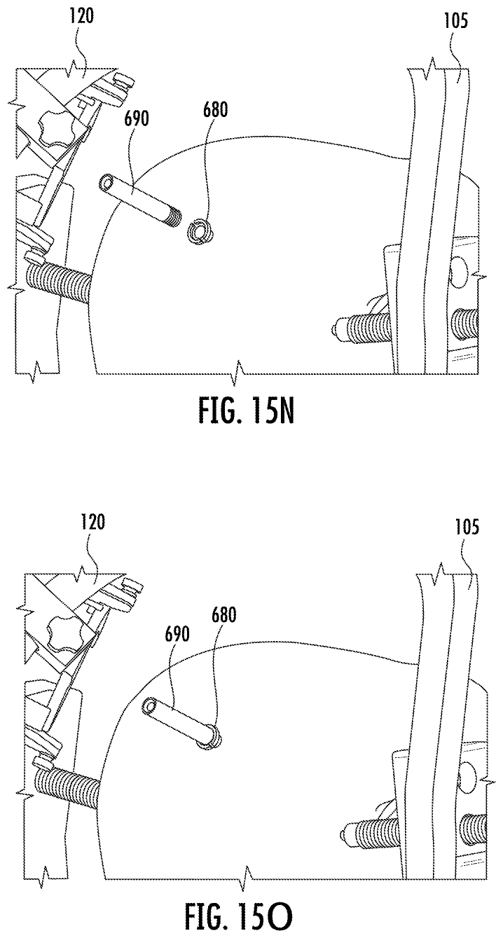

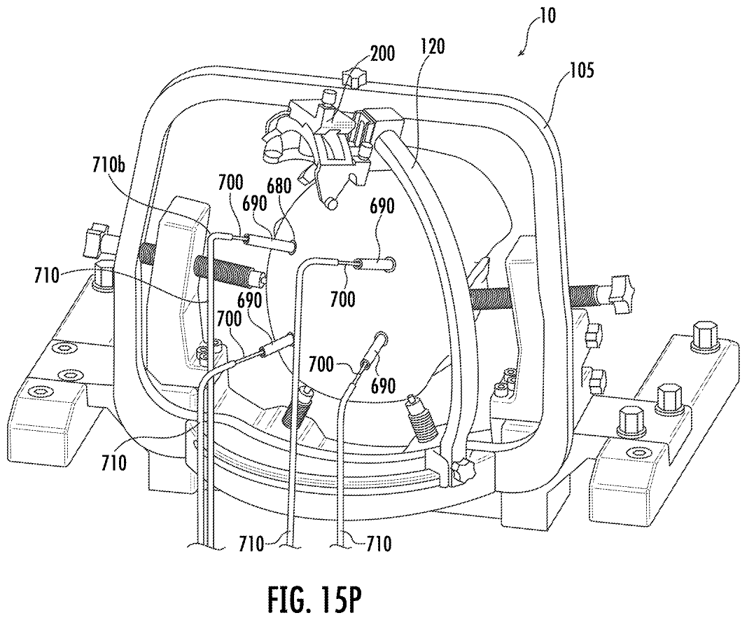

FIGS. 15A-15P illustrate an example workflow of an example neurological procedure that can be accommodated by the surgical tool system 10 of the present invention. FIG. 15A illustrates that multiple grids G can be used to identify respective multiple target entry sites S to a target intrabrain location(s). FIG. 15B illustrates four entry sites S₁-S₄ identified by four grids G₁-G₄, respectively. The grid G can be used with automated planning software to help define the appropriate trajectory path(s) and target site(s) in the body. See, e.g., U.S. Pat. No. 8,195,272, the contents of which are hereby by incorporated by reference as if recited in full herein. As shown in FIGS. 15A and 15B, the grids G and sites S can be identified before attaching the support arm 120 to the frame 105 of the frame mount assembly 100.

FIG. 15C shows that the support arm 120 can be attached to the base 115 and the frame 105 once the sites S are identified. FIG. 15D illustrates that the base 212 of the trajectory guide 201 as the surgical tool 200 can then be coupled to the support arm 120 via attachment member 230. However, the base 212 can be pre-assembled to the support arm 120 before the support arm 120 is coupled to the frame 105 or the base 115.

FIGS. 15D and E illustrate that the positional adjustments can be made by sliding the base 212 upward on the support arm 120 to align the tool 200 over a selected entry site S.

FIG. 15F illustrates that the trajectory guide body can be attached to the base 212 and the trajectory guide can be positionally adjusted using actuators 205 and/or the attachment member 230 using automated trajectory alignment and MRI and/or CT images.

FIGS. 15G and 15H illustrate that once the trajectory guide 201 is in a desired position, a drill guide 600 can be coupled to the trajectory guide 201. FIGS. 15I and 15J illustrate that a drill 650 can then be inserted into the drill guide 600 and a patient access hole can be drilled through the entry point/site S. Further details of a suitable MRI-compatible hand-held drill are described in U.S. Pat. No. 9,192,393, the contents of which are hereby incorporated by reference as if recited in full herein.

FIGS. 15K-15M show that a driver 660 can be used to secure a bushing 680 to the skull at the drilled hole location using the trajectory guide 201.

FIGS. 15N and 15O illustrate that a guide cylinder 690 can be secured to the bushing 680.

The process of navigating, drilling, and inserting additional bushings 680 can be carried out for the remaining (shown as three) entry points. Typically, once all bushings 680 are inserted, respective guide cylinders 690 can be attached thereto.

An intrabody fluid transfer device 700 is inserted into a guide cylinder 690. The intrabody fluid transfer device 700 can comprise an infusion cannula. The intrabody transfer device 700 can be coupled to flexible tubing 710. The infusion cannula can comprise a rigid MRI-compatible body. See, e.g., U.S. Pat. No. 10,105,485 and US Patent Application Publication Serial No. US 2017/0232229, the contents of which are hereby incorporated by reference as if recited in full herein.

The workflow can be adjusted to accommodate different procedures and/or patients.

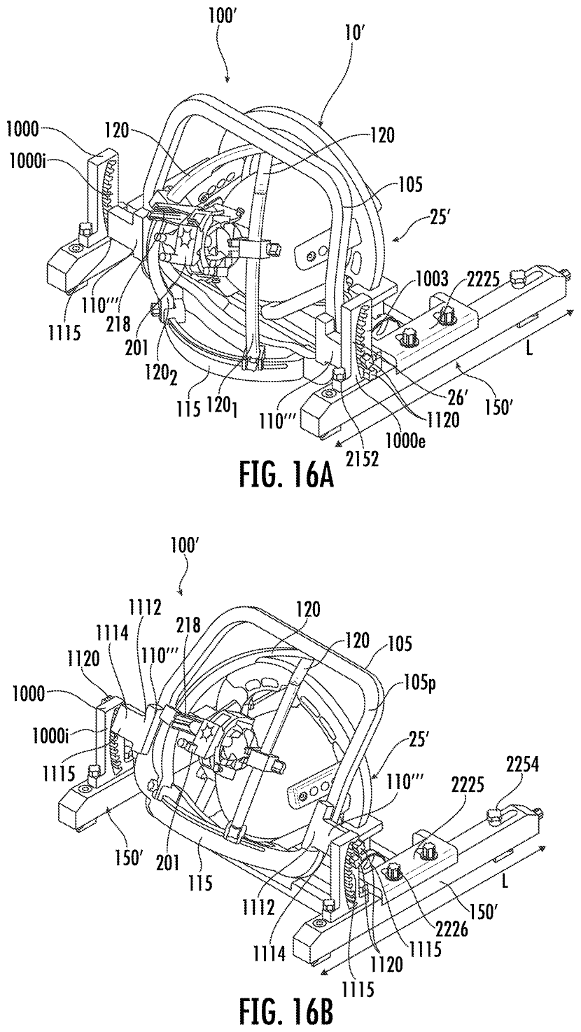

Referring now to FIGS. 16A and 16B, the surgical tool support system 10′ comprises another embodiment of a head fixation assembly 25′ coupled to a surgical frame mount assembly 100′. The head fixation assembly 25′ can be an adjustable cradle assembly comprising a ring 25r. For further description of the head fixation assembly 25′, see, U.S. Patent Application Publication Number 2020/0345572 by Monteris Medical Corporation, Plymouth, Minn., the contents of which are incorporated by reference as if recited in full herein.

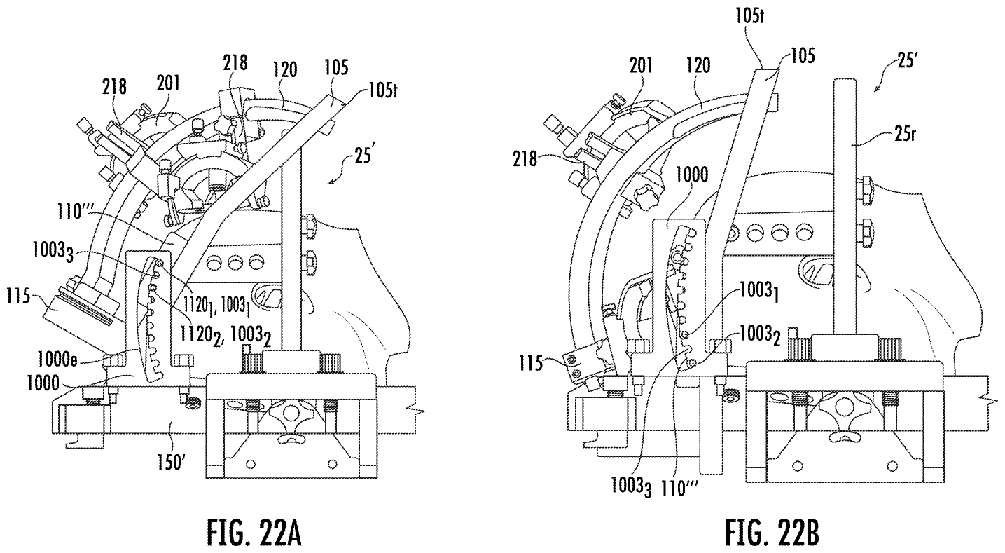

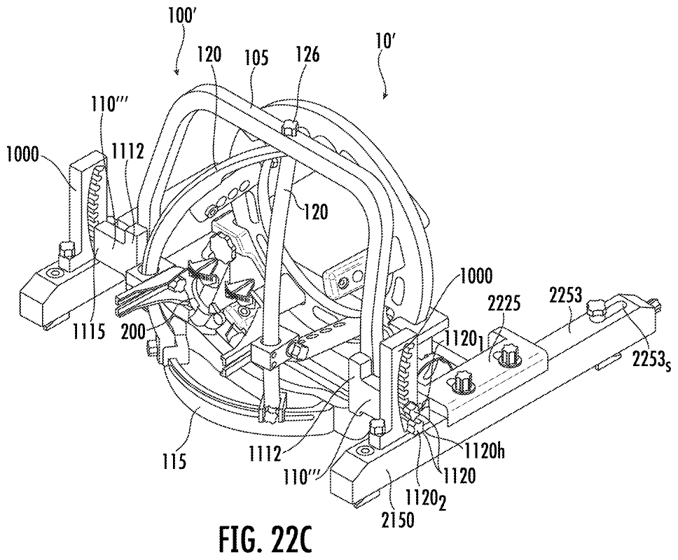

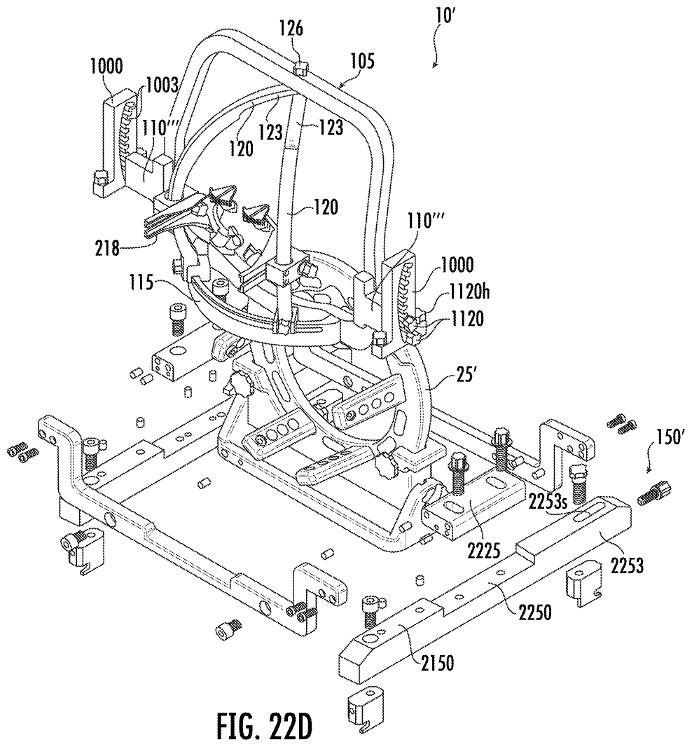

The surgical frame mount assembly 100′ can include at least one support arm 120, shown as first and second support arms 120₁, 120₂, attached to the frame member 105 and the base 115. The first and second support arms 120₁, 120₂ can be configured to hold respective first and second trajectory guides 201 for bilateral procedures. The base 115, the frame member 105, and the support arms 120 can be movable and/or pivotable as a unit up and down relative to the patient support table 50, the orientation plate 1000 and/or the head fixation assembly 25′ between at least a first orientation (first locked position) and a second orientation (second locked position). In a first orientation (FIG. 22A) the base 115 can reside a distance above a first orientation/use position with the top 105t of the frame member 105 forward relative to a second orientation (FIG. 22B) and with the top 105t above a head H of the patient.

The frame member 105 can be attached to coupling arms 110′″ extending off right and left sides thereof. Each coupling arm 110′″ can be configured to engage a corresponding support/side member 26′ which can be provided as an upright orientation plate 1000. The support member 26′ can reside on a table mount 150′ and extend upwardly therefrom.

As shown, the orientation plate 1000 comprises an elongate primary channel 1002 that merges into a plurality of secondary (smaller and/or shorter) channels 1003 that extend longitudinally along a length dimension “L” and define different lockable positions for the coupling arm 110′″ for orienting and holding the base 115, the support frame 105 and the support arm 120 as a unit in a desired selectable position. The plurality of secondary channels 1003 can be stacked one above another. The primary channel 1002 can be bounded by a wall 1002w with an arcuate shape (arcuate in a vertical/height dimension “h” as shown in FIG. 19) facing and across from the secondary channels 1003. The secondary channels 1003 can extend in the length dimension “l” as shown in FIG. 19.

Referring to FIGS. 16A, 16B, 19 and 21, neighboring secondary channels 1003 can be parallel and spaced apart a distance “d” in a height direction (measured from centerlines thereof) that is typically in a range of about 0.1 inch and about 1 inch. The secondary channels 1003 can be evenly spaced apart or unevenly spaced apart in a height dimension. At least one of the lower positioned secondary channels 1003 can have a closed end 1003e that angles up relative to an open entry end 1003o that is adjacent the primary channel 1002 while one or more of the upper positioned secondary channels 1003 can have a closed end 1003e that angles down. In some embodiments, the number of secondary channels can be in a range of 3-30, such as 3-20, 4-15, and 5-12 shown as 10.

The coupling arms 110′″ can be interchangeably referred to as “angle plates” as they can be oriented at different angles with respect to the orientation plate 1000, ring 25r of the head fixation assembly 25′ and/or patient for different lockable positions.

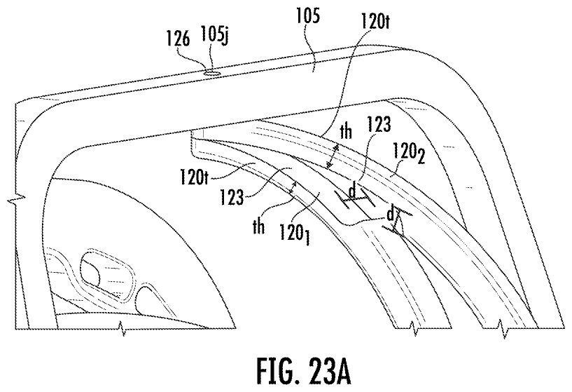

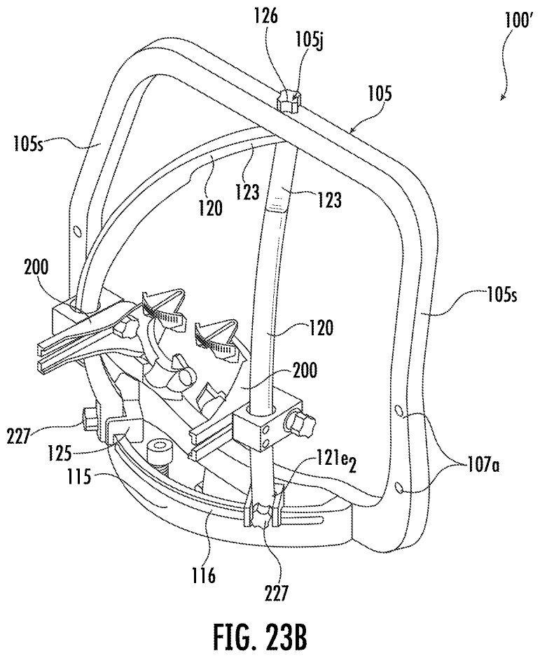

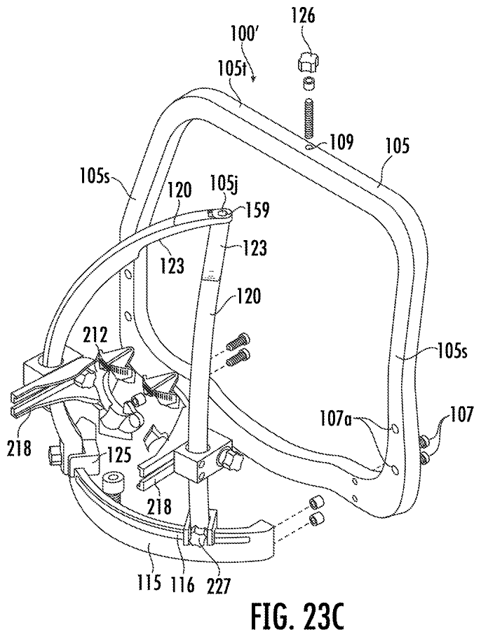

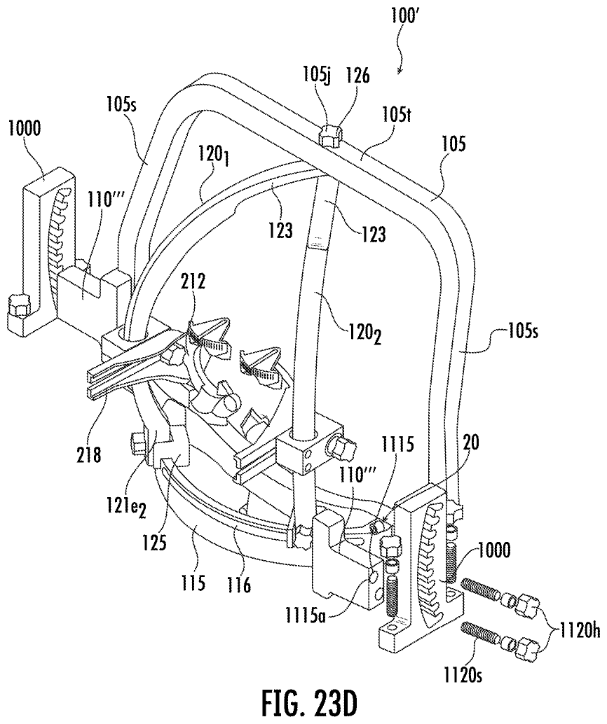

Referring to FIGS. 16A, 16B, 18, 21 and 22A-22D, the coupling arms 110′ can have a planar inner end segment 1112 that is secured to and that can abut a planar primary surface 105p of the frame member 105. Referring to FIGS. 21 and 23C, attachment members 107 can extend through apertures 107a in the frame member 105 into the inner end segment 1112 to couple the coupling arm 110′″ to the frame member 105.

The coupling arms 110′″ can have a body segment 1114 that extends laterally outward from the planar end segment 1112. The body segment 1114 can merge into an outer facing end segment 1115 that can couple to at least one attachment member 1120.

The coupling arms 110′″ can have a “T” shape with the head of the T turned 90 degrees and oriented to define the planar inner end segment 1112. However, other shapes may be used. In the embodiment shown, there are two attachment members 1120 that each engage a respective orientation plate 1000 and corresponding coupling arm 110′″. Each attachment member 1120 can concurrently reside in a different one of the secondary channels 1003 of a respective orientation plate 1000 to lock in position to provide the desired orientation of the support frame assembly 100″.

Referring to FIGS. 22A-22D, in some embodiments, a first attachment member 1120₁ engages a first secondary channel 1003₁ and a second attachment member 1120₂ engages a second secondary channel 1003₂ with a third secondary channel 1003₃ therebetween remaining open and devoid of any attachment member 1120.

Referring again to FIGS. 16A, 16B, the secondary channels 1003 are stacked one above another and can extend off the primary channel 1002 in a length dimension “L” of the table mount 150′ and/or table 50 (FIG. 1C) with the open end 1003o closer to a longitudinally positioned top of the head of a patient than the closed end 1003c.

Referring to FIG. 23D, each coupling arm 110′″ can extend laterally outward from the frame member 105. The outer end 1115 can terminate adjacent an inner surface 1000i of the orientation plate 1000. The outer end 1115 of the coupling arm 110′″ can comprise at least one aperture 1115a. The shaft 1120s can extend into one aperture 1115a and through a corresponding selected channel 1003 of the orientation plate 1000. Each attachment member 1120 can have a shaft 1120s that is slidably received in the primary channel 1002 and can slidably move up or down in the primary channel 1002 to enter a selected secondary channel 1003. A head 1120h of the attachment member 1120 can reside adjacent an external surface 1000e of the orientation plate 1000 at the selected secondary channel 1003 to lock the coupling arm 110′″ in position. The head 1120h can be detachable from the shaft 1120s. The shaft 1120s can be threaded and threadably engage the detachable head 1120h and the aperture 1115a.

Referring to FIGS. 18, 21 and 23D, the aperture(s) 1115a and 1112a can be cylindrical. The apertures 1115a in the outer facing end 1115 can be perpendicular to the apertures 1112a in the inner end segment 1112.

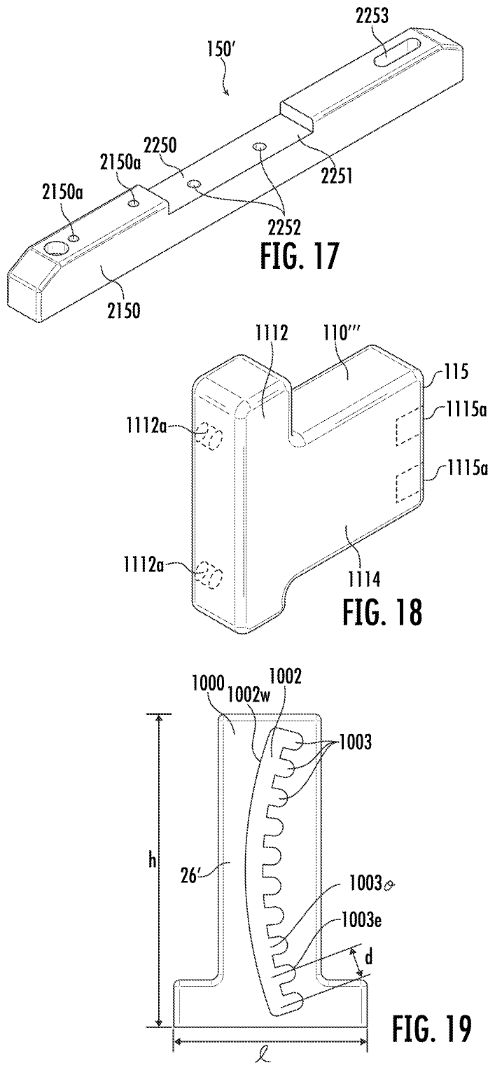

Referring to FIGS. 16A, 16B, 17, 21, 22C and 22D, the table mount 150′ can have a first section 2150 that receives the orientation plate 1000, a second section 2250 that receives a head fixation bracket 2225, and a third section 2253 that has a slot 2253s that receives a fixation member 2254 couples to the table 50 of the imaging system. The first section 2150 can include apertures 2150a that receive attachment members 2152 for attaching the orientation plate 1000. The second section 2250 may comprise a recessed segment 2251 with apertures 2252 for receiving fixation members 2226 that couple the bracket 2225 thereto (FIGS. 16A, 21). The recessed segment 2251 can reside below the outer surfaces of both the first and third sections 2150, 2253.

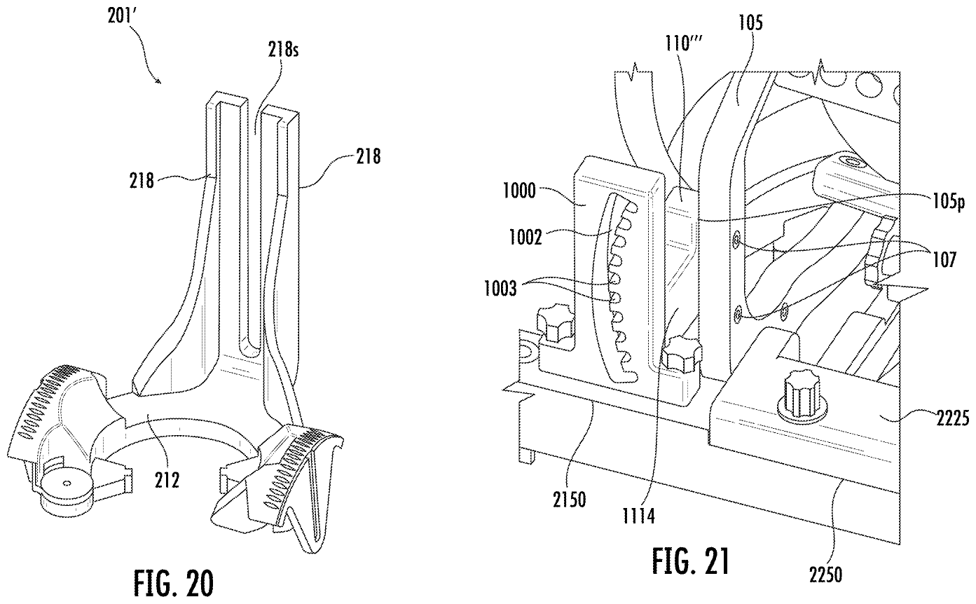

Referring to FIGS. 16A, 16B, 20, 22A, 22B, the surgical frame mount assembly 100′ can be configured to hold a trajectory guide 201 with a trajectory frame base 212 and an elongate leg 218 having an elongate slot 218s. In this embodiment, the slot 218s is open at the top of the elongate leg 218. The trajectory guide 201 can be slidably adjusted in position relative to the support arm 120 using the elongate leg 218.

Referring to FIGS. 22A, 22B and 23A-23D, the support legs 120₁, 120₂ can be configured to allow one arm 120₂ to slide over the other 120₁. As shown, both arms 120₁, 120₂ can be coupled to a common pivot joint 105j at a top of the frame member 105 and to separate locations of the base 115. Each arm 120₁, 120₂ can rotate independently of the other about the joint 105j. A top portion of segment 120t of each arm 120₁, 120₂ can have a stepped segment 123 defined by a thinner segment that has a first thickness “th” that merges into a thicker segment having a greater second thickness “th”, a distance below the top segment 120t. The stepped segment 123 of the top arm 120₂ can face down and the stepped segment 123 of the lower arm 120₁ can face up. Each stepped segment 123 can be aligned when one arm 120₂, is positioned over the other 120₁. The stepped segment 123 can extend about an arcuate sub-segment of the support arm 120.

FIGS. 22C and 22D illustrate the surgical system 10′ with the frame mount assembly 100′. FIGS. 23B-23D illustrate the frame mount assembly 100′ part of the surgical system 10′. The lower end portion 121e₂ of the support arm 120 can be coupled to a lower attachment member 227 that attaches to the base 115. The attachment member 227 can include or be coupled to a block member 125 that slidably couples to the base 115. The block member 125 can engage a slot 116 in the base 115 to pivot the arm 120 about the upper attachment member 227 laterally along a length of the base 115 to position the arm in different positions relative to the sides 105s of the frame member 105.

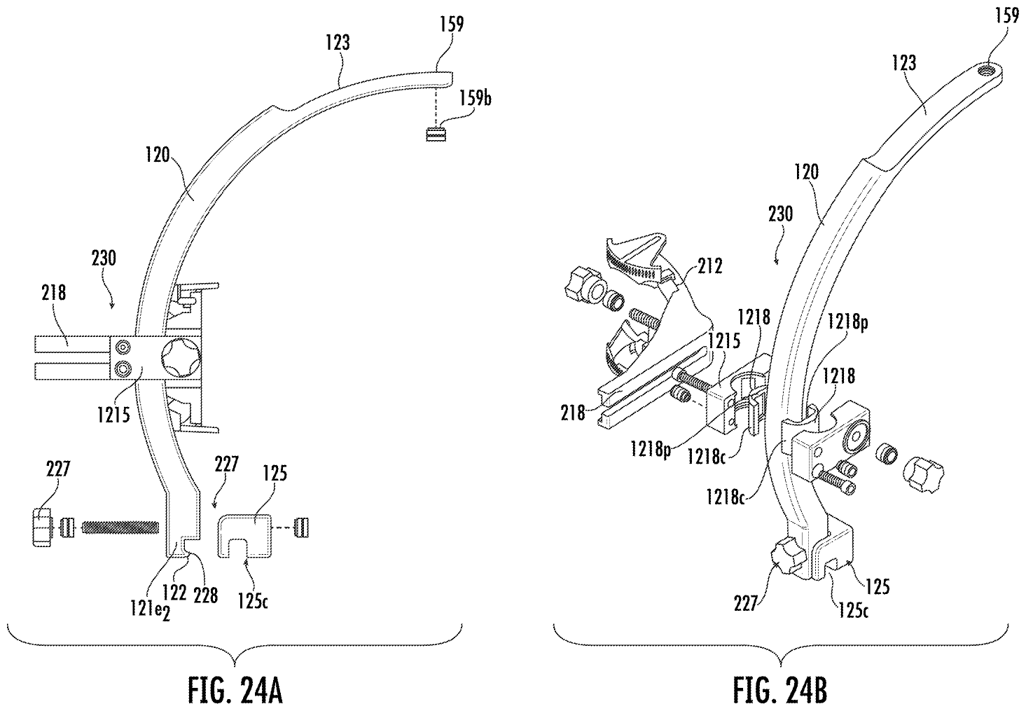

Referring to FIGS. 23C, 23D, 24A, 24B, the frame member 105 can have an aperture 109 medially located in the top 105t that receives the fixation member 126 and defines the pivot joint 105j. The arms 120 can each have an aligned aperture 159 in the top that comprise a bushing 159b that receives the fixation member 126 allowing the arms 120 to independently rotate about the pivot joint 105j.

FIGS. 24A, 24B illustrate that the attachment member 227 can comprise a block 125 with a lower, downwardly extending channel 125c that slidably couples to an upper portion of the base 115. The lower end portion 121e₂ of the arm 120 can have an inwardly extending ledge 228 that engages the slot 116 of the base 115.

FIGS. 24A and 24B also illustrate the attachment assembly 230 that can be used to attach the base 212 of the trajectory frame to the support arm 120 using the legs 218. The attachment assembly 230 can include a clamp assembly 1215 with an inner collar 1218 with a semi-circular outer wall 1218c and an inner surface with a planar channel 1218p. The clamp assembly 1215 is configured to clamp the collar 1218 against the support arm 120 to lock the base 212 of the trajectory frame in a selected position.

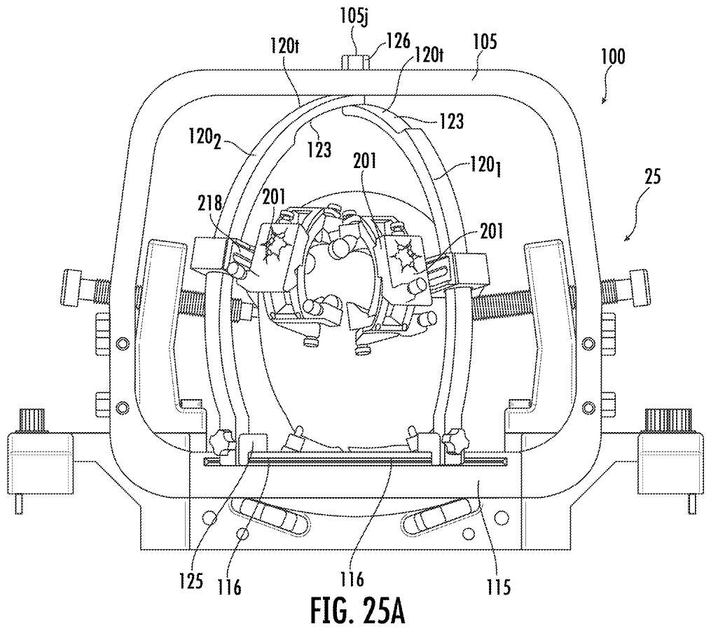

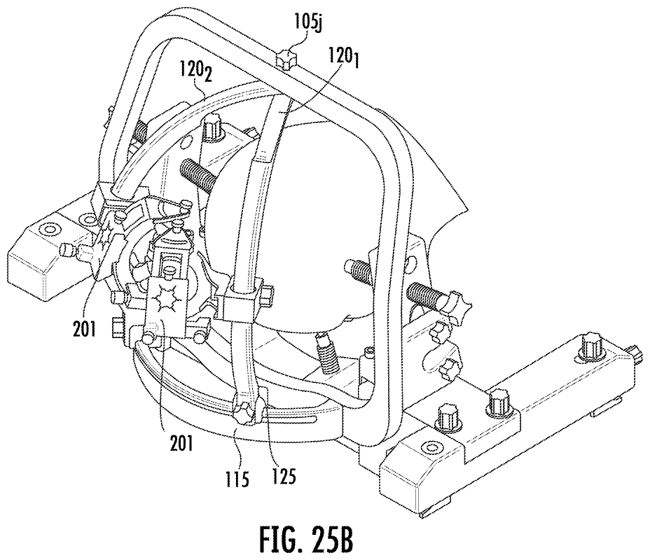

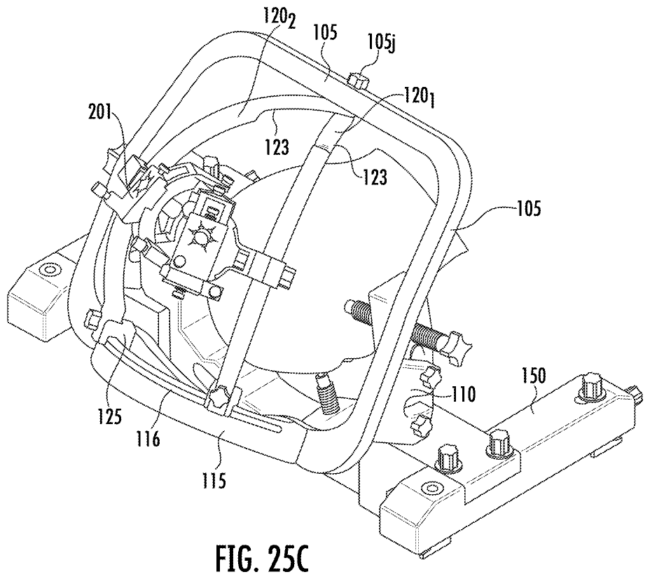

FIGS. 25A-25C illustrate that the surgical frame mount assembly 100 with the head fixation assembly 25 (FIGS. 1A-1C) can comprise the first and second support arms 120₁, 120₂ that can hold first and second trajectory guides 201, respectively, discussed with respect to the surgical frame mount assembly 100′.

The systems and assemblies can be configured to support initiating infusion in one or more regions while navigating and placing cannulae in others.

The surgical systems comprising the surgical tool support system can provide image-assisted navigation, high accuracy, and safety.

Embodiments of the invention can provide for multiple trajectories and multiple simultaneous infusions. The surgical tool support system 10, 10′ and/or the surgical frame mount assembly 100, 100′ and tools 200, such as trajectory guides 201, can provide for trajectory adjustments in all degrees of freedom.

Embodiments of the invention provide trajectory guides 201 and support frame assemblies 100, 100′ that require no direct attachment to the skull.

For multiple entry sites, a single trajectory guide 201 can be used to place multiple devices during a single treatment session that may be less expensive over known conventional systems.

The surgical tool support systems 10, 10′ can be sufficiently rigid and secure to provide mechanical stability for drilling.

The surgical tool support systems 10, 10′ can be configured for all-MRI, or OR+MRI procedures or CT and MRI or just CT procedures.

Embodiments of the invention do not mount the trajectory-frame 212 to the skull unlike conventional stereotactic systems.

Embodiments of the invention may be particularly suitable for multi-delivery (concurrent infusion) workflows.

In some embodiments, a substance can be delivered to the target region(s) through the delivery device 700 (FIG. 15P) may be any suitable and desired substance(s). According to some embodiments, the substance is a liquid or slurry. In the case of a tumor, the substance may be a chemotherapeutic (cytotoxic) fluid. In some embodiments, the substance can include certain types of advantageous cells that act as vaccines or other medicaments (for example, antigen presenting cells such as dendritic cells). The dendritic cells may be pulsed with one or more antigens and/or with RNA encoding one or more antigen. Exemplary antigens are tumor-specific or pathogen-specific antigens. Examples of tumor-specific antigens include, but are not limited to, antigens from tumors such as renal cell tumors, melanoma, leukemia, myeloma, breast cancer, prostate cancer, ovarian cancer, lung cancer and bladder cancer. Examples of pathogen-specific antigens include, but are not limited to, antigens specific for HIV or HCV. In some embodiments, the substance may comprise radioactive material such as radioactive seeds. Substances delivered to a target site(s) may include, but are not limited to, the following as shown in Table 1:

TABLE 1

| DRUG (generic name) | DISORDER(S) |
|---|---|
| Caprylidene | Alzheimer's disease |
| Donepezil | Alzheimer's disease |
| Galantamine | Alzheimer's disease |
| Memantine | Alzheimer's disease |
| Tacrine | Alzheimer's disease |
| vitamin E | Alzheimer's disease |
| ergoloid mesylates | Alzheimer's disease |
| Riluzole | Amyotrophic lateral sclerosis |
| Metoprolol | Benign essential tremors |
| Primidone | Benign essential tremors |
| Propanolol | Benign essential tremors |
| Gabapentin | Benign essential tremors & Epilepsy |
| Nadolol | Benign essential tremors & Parkinson's disease |
| Zonisamide | Benign essential tremors & Parkinson's disease |
| Carmustine | Brain tumor |
| Lomustine | Brain tumor |
| Methotrexate | Brain tumor |
| Cisplatin | Brain tumor & Neuroblastoma |
| Ioversol | Cerebral arteriography |
| Mannitol | Cerebral Edema |
| dexamethasone | Cerebral Edema & Neurosarcoidosis |
| Baclofen | Cerebral spasticity |
| Ticlopidine | Cerebral thrombosis/embolism |
| isoxsuprine | Cerebrovascular insufficiency |
| cefotaxime | CNS infection & Meningitis |
| Acyclovir | Encephalitis |
| Foscarnet | Encephalitis |
| ganciclovir | Encephalitis |
| interferon alpha-2a | Encephalitis |
| carbamazepine | Epilepsy |
| clonazepam | Epilepsy |
| Diazepam | Epilepsy |
| divalproex sodium | Epilepsy |
| ethosuximide | Epilepsy |
| Ethotoin | Epilepsy |
| Felbamate | Epilepsy |
| fosphenytoin | Epilepsy |
| levetiracetam | Epilepsy |
| mephobarbital | Epilepsy |
| paramethadione | Epilepsy |
| Phenytoin | Epilepsy |
| trimethadione | Epilepsy |
| Pregabalin | Epilepsy & Neuralgia |
| immune globulin | Guillain-Barre Syndrome |
| intravenous | |
| interferon beta-1b | Guillain-Barre Syndrome & Multiple sclerosis |
| azathioprine | Guillain-Barre Syndrome & Multiple sclerosis & |
| | Neurosarcoidosis |
| risperidone | Head injury |
| tetrabenazine | Huntington's disease |
| acetazolamide | Hydrocephalus & Epilepsy |
| Alteplase | Ischemic stroke |
| clopidogrel | Ischemic stroke |
| nimodipine | Ischemic stroke & Subarachnoid hemorrhage |
| Aspirin | Ischemic stroke & Thromboembolic stroke |
| Amikacin | Encaphalitis |
| Ampicillin | Encaphalitis |
| ampicillin/sulbactam | Encaphalitis |
| ceftazidime | Encaphalitis |
| ceftizoxime | Encaphalitis |
| cefuroxime | Encaphalitis |
| chloramphenicol | Encaphalitis |
| cilastatin/imipenem | Encaphalitis |
| gentamicin | Encaphalitis |
| meropenem | Encaphalitis |
| metronidazole | Encaphalitis |
| Nafcillin | Encaphalitis |
| Oxacillin | Encaphalitis |
| piperacillin | Encaphalitis |
| Rifampin | Encaphalitis |
| sulfamethoxazole/ | Encaphalitis |
| trimethoprim | |
| tobramycin | Encaphalitis |
| triamcinolone | Encaphalitis |
| vancomycin | Encaphalitis |
| ceftriaxone | Encaphalitis & Neurosyphilis |
| Penicillin | Encaphalitis & Neurosyphilis |
| corticotropin | Multiple sclerosis |
| dalfampridine | Multiple sclerosis |
| Glatiramer | Multiple sclerosis |
| mitoxantrone | Multiple sclerosis |
| natalizumab | Multiple sclerosis |
| Modafinil | Multiple sclerosis |
| cyclophosphamide | Multiple sclerosis & Brain tumor & |
| | Neuroblastoma |
| interferon beta-1a | Multiple sclerosis & Neuritis |
| prednisolone | Multiple sclerosis & Neurosarcoidosis |
| prednisone | Multiple sclerosis & Neurosarcoidosis |
| amantadine | Multiple sclerosis & Parkinson's disease |
| methylprednisolone | Neuralgia |
| desvenlafaxine | Neuralgia |
| nortriptyline | Neuralgia |
| doxorubicin | Neuroblastoma |
| Vincristine | Neuroblastoma |
| albendazole | Neurocystecercosis |
| chloroquine phosphate | Neurosarcoidosis |
| hydroxychloroquine | Neurosarcoidosis |
| Infliximab | Neurosarcoidosis |
| pentoxyfilline | Neurosarcoidosis |
| thalidomide | Neurosarcoidosis |
| apomorphine | Parkinson's disease |
| belladonna | Parkinson's disease |
| benztropine | Parkinson's disease |
| Biperiden | Parkinson's disease |
| bromocriptine | Parkinson's disease |
| Carbidopa | Parkinson's disease |
| carbidopa/entacapone/ | Parkinson's disease |
| levodopa | |
| carbidopa/levodopa | Parkinson's disease |
| entacapone | Parkinson's disease |
| Levodopa | Parkinson's disease |
| pergolide mesylate | Parkinson's disease |
| pramipexole | Parkinson's disease |
| procyclidine | Parkinson's disease |
| Rasagiline | Parkinson's disease |
| Ropinirole | Parkinson's disease |
| Rotiotine | Parkinson's disease |
| scopolamine | Parkinson's disease |
| Tolcapone | Parkinson's disease |
| trihexyphenidyl | Parkinson's disease |
| Seleginline | Parkinson's disease |
| rivastigmine | Parkinson's disease & Alzheimer's disease |
| anisindione | Thromboembolic stroke |
| Warfarin | Thromboembolic stroke |
| 5-hydroxytryptophan | Depression & Anxiety & ADHD |
| Duloxetine | Depression & Anxiety & Bipolar disorder |
| escitalopram | Depression & Anxiety & Bipolar disorder |
| venlafaxine | Depression & Anxiety & Bipolar disorder & |
| | Autism & Social anxiety disorder |
| desvenlafaxine | Depression & Anxiety & PTSD & ADHD |
| Paroxetine | Depression & Anxiety & PTSD & Social anxiety |
| | disorder |
| fluoxetine/olanzapine | Depression & Bipolar disorder |
| l-methylfolate | Depression & BPD |
| amitriptyline | Depression & PTSD |
| Sertraline | Depression & PTSD & Bipolar disorder & Social |
| | anxiety disorder |
| fluvoxamine | Depression & PTSD & Social anxiety disorder |
| Olanzapine | Depression & Schizophrenia & Bipolar disorder |
| paliperidone | Depression & Schizophrenia & Bipolar disorder |
| aripiprazole | Depression & Schizophrenia & Bipolar disorder |
| | & Autism |
| Quetiapine | Depression & Schizophrenia & PTSD & BPD & |
| | Bipolar disorder |
| risperidone | Depression & Schizophrenia & PTSD & BPD & |
| | Bipolar disorder & Autism |
| amisulpride | Depression & Social anxiety disorder |
| chlorpromazine | Psychosis |
| Droperidol | Psychosis |
| fluphenazine | Psychosis |
| periciazine | Psychosis |
| perphenazine | Psychosis |
| thiothixene | Psychosis |
| triflupromazine | Psychosis |
| haloperidol | Psychosis & Dementia |
| Prazosin | PTSD |
| Clozapine | Schizophrenia |
| flupenthixol | Schizophrenia |
| iloperidone | Schizophrenia |
| Loxapine | Schizophrenia |
| mesoridazine | Schizophrenia |
| Promazine | Schizophrenia |
| Reserpine | Schizophrenia |
| thioridazein | Schizophrenia |
| zuclopenthixol | Schizophrenia |
| Asenapine | Schizophrenia & Bipolar disorder |
| levomepromazine | Schizophrenia & Bipolar disorder |
| ziprasidone | Schizophrenia & Bipolar disorder |
| Molindone | Schizophrenia & Psychosis |
| Pimozide | Schizophrenia & Psychosis |
| thioridazine | Schizophrenia & Psychosis |
| Cytarabine | Chemotherapy, hematological malignancies |

According to some embodiments, the surgical cannula is used to remove or withdraw a substance therethrough from the target area.

Embodiments of the present invention may include steps, features, aspects, components, procedures and/or systems as disclosed in U.S. patent application Ser. No. 12/236,854, published as U.S Published Patent Application No. 2009/0171184, the disclosure of which is incorporated herein by reference. Embodiments of the present invention use the surgical support system 10 with an automated or semi-automated surgical navigation system comprising defined workflows and DICOM communication with an MR Scanner. See, e.g., U.S. Pat. No. 10,105,485, the contents of which are hereby incorporated by reference as if recited in full herein.

According to some embodiments, the systems are configured to provide a substantially automated or semi-automated and relatively easy-to-use MM-guided system with defined workflow steps and interactive visualizations. In particular embodiments, the systems define and present workflow with discrete steps for finding target and entry point(s), guiding the alignment of the targeting cannula to a planned trajectory, monitoring the insertion of the delivery cannula, and adjusting the (X-Y) position in cases where the placement needs to be corrected. During steps where specific MR scans are used, the circuit or computer module can display data for scan plane center and angulation to be entered at the console. The workstation/circuit can passively or actively communicate with the MR scanner. The system can also be configured to use functional patient data (e.g., fiber tracks, fMRI and the like) to help plan or refine a target surgical site and/or access path.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the teachings and advantages of this invention. Accordingly, all such modifications are intended to be included within the scope of this invention as defined in the claims. The invention is defined by the following claims, with equivalents of the claims to be included therein.

That which is claimed is:

1. A surgical tool support system comprising:
a frame member comprising a perimeter with a top, bottom and right and left sides surrounding an open space;
a base coupled to the bottom of the frame member;
at least one support arm having opposing first and second ends, the first end coupled to the top of the frame member, the second end coupled to the base, wherein the at least one support arm extends across the open space between the top and the bottom of the frame member; and
first and second coupling arms, the first coupling arm attached to the right side of the frame member, the second coupling arm attached to the left side of the frame member,
wherein the frame member, the base and the at least one support arm are configured to move as a unit between one of a plurality of different use orientations to thereby allow supine and occipital procedures.

2. The system of claim 1, further comprising a first side support member attached to a first table mount assembly and a second side support member attached to a second table mount assembly, wherein the first coupling arm is attached to the first side support member and the second coupling arm is attached to the second side support member.

3. The system of claim 1, wherein the first and second side support members are first and second orientation plates that each comprise a primary channel extending upwardly and a plurality of secondary channels extending from the primary channel and being spaced apart in a vertical direction.

4. The system of claim 1, wherein the base is arcuate and arcs outward in a direction that is longitudinally spaced apart from a bottom of the frame member, wherein the base comprises an arcuate laterally extending groove that extends across an upper portion thereof, and wherein a lower end portion of the support arm is adjustably lockable to the base.

5. The system of claim 1, wherein the at least one support arm is pivotably coupled to a medial segment of the top of the frame member.

6. The system of claim 1, further comprising a trajectory guide assembly attached to the at least one support arm, wherein the trajectory guide assembly comprises a trajectory frame with a pair of adjacent legs on opposing sides of a slot that is slidably positionable at different positions above and/or below the at least one support arm.

7. The system of claim 1, wherein the at least one support arm is provided as first and second support arms each separately positionable relative to each other and the base and each coupled to a common attachment location at the top of the frame member.

8. The system of claim 7, wherein the first support arm has an upper portion with a downwardly extending recess, wherein the second support arm has an upper portion with an upwardly extending recess, and wherein the first and second support arms cooperate to allow the first support arm to slide over the second support arm.

9. The system of claim 3, wherein the secondary channels have a closed end and an opposing open end, and wherein the open end is adjacent to and merges into the primary channel.

10. The system of claim 3, wherein the primary channel is bounded by a wall that is arcuate in a vertical/height dimension.

11. The system of claim 10, wherein the wall has upper and lower ends that are more distally positioned relative to a medial segment thereof.

12. The system of claim 3, wherein the plurality of secondary channels are provided as between 3 and 30 secondary channels.

13. The system of claim 12, wherein neighboring secondary channels of the plurality of secondary channels are spaced apart in a vertical direction a distance in a range of about 0.1 inch to 1 inch.

14. The system of claim 3, wherein the first and second coupling arms each have a first end portion and a laterally spaced apart second end portion, wherein the first end portion of the first coupling member is attached to the first side of the frame member and the first end portion of the second coupling member is attached to the second side of the frame member, wherein the second end portion of the first coupling member is attached to at least one laterally extending attachment member, each of which also extends through one of the secondary channels of the first orientation plate, and wherein the second end portion of the second coupling member is attached to at least one laterally extending attachment member, each of which also extends through one of the secondary channels of the second orientation plate.

15. A method for positioning a surgical tool about a head of a patient, the method comprising:
providing a frame mount assembly comprising a frame member having a perimeter with a top, bottom and right and left sides surrounding an open space and at least one support arm that extends across the open space between the top and the bottom of the frame member, wherein the open space is larger laterally and vertically than the head of the patient; and
moving the at least one support arm and the frame member as a unit between one of a plurality of different use orientations, wherein the frame mount assembly is configured to allow a user to select at least one orientation for a supine procedure and/or at least one orientation for an occipital procedure.

16. The method of claim 15, wherein the frame mount assembly further comprises a base coupled to the bottom of the frame member, wherein the at least one support member has a first end portion attached to a medial segment of the top of the frame member and a second end portion attached to the base, the method further comprising pivoting the first end portion relative to the medial segment of the top of the frame while sliding the second end portion over the base and locking the second end portion in a desired orientation relative to the base before or after the moving step.

17. The method of claim 15, wherein the frame mount assembly comprises left and right coupling arms that attach to left and right side support members that are coupled to a table mount assembly, and wherein the moving step comprises mounting the left and right coupling arms to a different coupling aperture or channel of the left and right side support members.

18. The method of claim 15, further comprising adjusting a position of a trajectory guide frame attached to the at least one support arm before, during or after the moving step.

19. The method of claim 15, further comprising attaching a head fixation frame assembly to a table mount assembly and attaching the frame member to the table mount assembly adjacent the head fixation frame assembly before the moving step.

20. The method of claim 15, further comprising placing multiple intrabody cannulas during a single treatment session for neurological treatment of a brain of the patient during an Mill and/or CT-image guided medical procedure.
